(19) United States
(12) Patent Application Publication
(10) Pub. No.: US 2023/0410294 A1
(43) Pub. Date: Dec. 21, 2023

(54) METHOD FOR GRADING THE NUCLEAR MORPHOLOGY OF A TUMOR

(71) Applicant: Nanyang Technological University, Singapore (SG)

(72) Inventors: Wenting ZHAO, Singapore (SG); Yongpeng ZENG, Singapore (SG); Aninda MITRA, Singapore (SG)

(21) Appl. No.: 18/036,078

(22) PCT Filed: Nov. 10, 2021
(86) PCT No.: PCT/SG2021/050687

§ 371 Date: May 9, 2023

(30) Foreign Application Priority Data
Nov. 10, 2020 (SG) 10202011177R

(51) Int. Cl. G06T 7/00

(52) U.S. Cl. CPC G06T 7/0012; G06T 2207/10056; G06T 2207/30024

(57) ABSTRACT

The present invention relates to a method of grading the nuclear morphology of a tumor cell. The method may comprise a) culturing the tumor cell on an array comprising a plurality of nanopillars; and b) assessing a deformation pattern of the nucleus, wherein an isotropic deformation indicates low malignancy of the tumor cell, while a linear or anisotropic deformation pattern indicates high malignancy of the tumor cell. In another embodiment, the method comprises determining a nanopillar-induced deformation profile comprising an angular distribution of orientations in a plurality of regions of interests (ROIs) of the image and a coherency in each ROI. The invention also relates to a method of predicting the likelihood of a metastatic cancer or predicting a response of a tumor to a cancer drug in a subject based on assessing the degree of nuclear deformation of a cell sample cultured on the surface comprising a plurality of nanopillars.

### FIELD OF INVENTION

The present invention relates to the field of cancer pathology and detection. In particular, the specification teaches a method of grading the nuclear morphology of a tumor cell.

### BACKGROUND

Tumor grading is a critical assessment in cancer pathology and has a strong influence on cancer diagnosis and treatment. Tumor grading evaluates the probability of tumor cells to migrate and invade to cause cancer metastasis, i.e. tumor malignancy. Accurate tumor grading supports both earlier tumor diagnosis and better monitoring the efficacy of cancer therapeutics and prognosis. Nevertheless, due to the complex mechanism of oncogenesis, it is extremely challenging to grade tumor aggressiveness as there are no universal genes or proteins to mark the progression of malignancy across different cancer types and individuals. Currently, the clinical practice for tumor grading involves histopathological diagnosis, where the pathologist relies primarily on visual inspection of a patient's biopsy sample using light microscopy in order to judge the morphological abnormalities in cells and tissues. Due to the huge diversity between cancer tissue types and individual patients, the grading result heavily depends on the experience of the pathologist, and thus highly subjective. Tools for objective and quantitative cancer grading are at high-demand.

In current clinical practice, evaluations of nuclear morphology have been successfully used to assess tumor malignancy, which is the major part of nuclear grading. Alteration in nuclear morphology is observed in many cancers, and its evaluation, i.e. nuclear grading, can be sufficient to diagnose and grade cancer, and predict prognosis. In tumor pathology assessment, nuclear changes are typically visualized by either hematoxylin and eosin (H&E) or Papanicolaou stained biopsy specimen using brightfield microscopy. Microscopically, nuclear size and shape are the most straightforward to measure, hence are commonly used as a quantitative parameter for different types of tissue samples including breast, bladder, cervix, colon, kidney, liver, lung, ovary, pancreas, prostate, skin, and thyroid cancers. While size is easy to measure, the large variations between individual cancer cells or even normal cells across different cancer types make it challenging to standardize. In comparison, the abnormalities in nuclear membrane morphology, such as the appearance of blebs, lobes, invaginations, or clefts, are distinct features of cancer cells. Researchers have found that the presence of irregular folds and invagination of the nuclear periphery significantly correlated with lymph node metastasis. In S′ezary disease (cutaneous T-cell lymphoma) or adult T-cell leukemia/lymphoma caused by human T-lymphotropic virus type 1 infection, nuclei were found in a flower shape. However, the evaluation of such diverse abnormalities is much challenging due to the randomness of the altered nuclear membrane structures in terms of both geometry and location. The current assessment method relies primarily on visual inspection of a patient's biopsy sample using light microscopy to grade the morphological alterations. Although confocal microscopy and 3D image reconstruction has been applied to enhance the accuracy in capturing the fine alterations in cancer cells' nuclei, the quantification of the nuclear deformations with irreproducible nature among individual cells is still a bottleneck preventing its standardization for clinical applications.

It would be desirable to overcome or ameliorate at least one of the above-described problems, or at least to provide a useful alternative.

### SUMMARY

Disclosed herein is a method of grading the morphology of a tumor cell, the method comprising:
a) culturing the tumor cell on an array comprising a plurality of nanopillars; and
b) assessing a deformation pattern of the nucleus to provide an indication of the morphology of the tumor cell.

Disclosed herein is a method of grading a sample of tumor cells, comprising:
a) obtaining an image of one or more cells of the sample captured on an array of nanopillars; and
b) determining a nanopillar-induced deformation profile of the one or more cells.

Disclosed herein is a method of predicting the progression of a tumor in a subject, the method comprising:
a) culturing a cell sample on a surface comprising a plurality of nanopillars; and
b) assessing the degree of nuclear deformation of cells of the cell sample to provide an indication of the progression of tumor in the subject.

In one embodiment, the degree of nuclear deformation may refer to the change in the pattern of nuclear deformation of cells of the cell sample as compared to a reference.

Disclosed herein is a method of predicting the likelihood of a metastatic cancer in a subject, the method comprising:
a) culturing a cell sample on a surface comprising a plurality of nanopillars; and
b) assessing the degree of nuclear deformation of cells of the cell sample to predict the likelihood of a metastatic cancer in a subject.

Disclosed herein is a method of predicting a response of a tumor to a cancer drug in a subject, the method comprising:
a) culturing a cell sample on a surface comprising a plurality of nanopillars; and b) assessing the degree of nuclear deformation of cells of the cell sample to predict the response of the tumor to the cancer drug in the subject.

### BRIEF DESCRIPTION OF DRAWINGS

Embodiments of the present invention are hereafter described, by way of non-limiting example only, with reference to the accompanying drawings in which:

FIG. 1. The workflow of nanopillar-based nuclear grading. a) Fabrication of nanochips. b) Guidance of the nuclear deformation in cancer cells. c) Fluorescence imaging and image analysis for statistical analysis.

FIG. 2. Fabrication of the nanostructured platform. (a) Fabrication procedures of nanochips. (b) SEM micrographs of nanopillar arrays with different diameters. Scale bar, 1 μm. (c) SEM micrographs of nanopillar arrays with different pitches. Scale bar, 1 μm.

FIG. 3. Nanopillar-induced morphological alterations in the U2OS nuclear envelope at different Z positions.

FIG. 4. One example showing the quantification matrix for characterization of nanopillar-guided subnuclear deformations under distinct conditions. a) Schematics of nanopillar-enabled characterization of subnuclear irregularities in cancer cells. b-d) Morphological changes in nanopillar-guided subnuclear irregularities in cells with different treatments, including (b) feature area, (c) feature length and (d) feature orientation variation. e) Fraction analysis of nuclear deformation patterns under different conditions. Statistical significance of the two groups was compared using an unpaired t test with Welch's correction, p-value: **P<0.01; *P<0.05; ns>0.05.

FIG. 5. Commercial applications: nanostructure-guided quantification of nuclear abnormalities for many nucleus-related diseases. This invention can be applied for both solid biopsy and liquid biopsy. By characterizing the nuclear deformation on nanochips, it can potentially enable digital classification of cancer stage, cancer therapy monitoring as well as anti-metastatic drug screening.

FIG. 6. Nanopillar-guided subnuclear deformation patterns correlate with cancer malignancy. a) Schematics of different patterns generated by nanopillar-guided nuclear shape deformation in cancer cells with varying malignancies. b) SEM of nanopillar arrays. Scale bar, 2 μm. c) Nuclear morphology of MCF-7 cells and MDA-MB-231 cells on a flat surface. Scale bars, 5 μm. d) Dynamics of nanopillar-guided nuclear features in MCF-7 and MDA-MB-231 cells for one hour. Red dots indicate nanopillar locations. Red arrows in the bottom row refer to the nanopillars that guide the nuclear grooves. Scale bars, 3 μm. e) Orientation of nuclear shape irregularities and nanopillar-guided nuclear features in MDA-MB-231 cells and MCF-7 cells. f) Comparison of orientation distribution of nuclear shape irregularities and nanopillar-guided nuclear features in MDA-MB-231 cells and MCF-7 cells. g) Anisotropy measurement of the nanopillar-guided nuclear deformation in MCF-7 cells (N=94 pillars) and MDA-MB-231 cells (N=44 pillars). h) Fraction of ring deformation and line deformation in MCF-7 cells (N=39 cells) and MDA-MB-231 cells (N=24 cells). Ring deformation is defined by c.c.<0.3 whereas ring deformation is defined by c.c.>0.3. Statistical significance of the two groups was compared using an unpaired t test with Welch's correction, p-value: ****<0.0001.

FIG. 7. Influence of nanopillar geometry on the subnuclear deformation patterns. a) SEM micrographs show nanopillar arrays with different diameters. Scale bar, 1 μm. b) Fluorescent images showing nuclear deformation patterns of MCF-7 cells and MDA-MB-231 cells on nanopillar arrays with different diameters. Scale bar, 5 μm. c) Anisotropy measurement of nanopillar-guided nuclear features in MDA-MB-231 and MCF-7 cells on nanopillar arrays with different diameters. (MDA-MB-231: N=47 pillars (D200); 61 pillars (D300); 85 pillars (D400); 44 pillars (D500); 58 pillars (D600); 54 pillars (D700); 41 pillars (D800); MCF-7: N=54 pillars (D200); 43 pillars (D300); 72 pillars (D400); 94 pillars (D500); 65 pillars (D600); 52 pillars (D700); 61 pillars (D800).). Pitch for all arrays is 3 μm. Error bars represent s.e.m. d) Fraction of cells showing ring deformation and line deformation in MDA-MB-231 cells and MCF-7 cells on nanopillar arrays with different diameters (MDA-MB-231: N=26 cells (D200); 32 cells (D300); 48 cells (D400); 24 cells (D500); 35 cells (D600); 36 cells (D700); 30 cells (D800); MCF-7: N=29 cells (D200); 26 cells (D300); 33 cells (D400); 39 cells (D500); 42 cells (D600); 40 cells (D700); 41 cells (D800).). Error bars represent SD. e) Top-view SEM images of nanopillar arrays with varying pitches (3 μm and 5 μm), and fluorescent images showing nuclear deformation patterns of MCF-7 cells and MDA-MB-231 cells on nanopillar arrays with different pitches. Scale bars, 5 μm. f) Anisotropy measurement of MDA-MB-231 and MCF-7 cells on nanopillar arrays with varied pitches (MDA-MB-231: N=42 pillars (P3); 27 pillars (P5); MCF-7: N=94 pillars (P3); 49 pillars (P5).). Diameter for all arrays is 500 nm. f) Fraction of cells showing ring deformation and line deformation for MDA-MB-231 cells and MCF-7 cells on nanopillar arrays with different pitches (MDA-MB-231: N=24 cells (P3); 27 pillars (P5); MCF-7: N=39 cells (P3); 49 pillars (P5).). Error bars represent SD. Statistical significance of coherency measurement under different conditions was evaluated by an unpaired t-test with Welch's correction. ****P<0.0001; **P<0.01; ns>0.05.

FIG. 8. Probing cancer heterogeneity via nanopillar-induced subnuclear deformation. a) Methodology for using nanopillar arrays to probe cancer heterogeneity. b) MDA-MB-231 cell and GFP-tagged MCF-7 cells showing different nuclear deformation patterns on the same substrate. Scale bar 10 μm. c) Correlation between fraction of cells showing line-like guided nuclear deformation and the ratio of MDA-MB-231 cells to MCF-7 cells. d) Brightfield images of MCF-7 cells and MDA-MB-231 cells migrating on nanopillar arrays over time and the fluorescent images showing deformation patterns of cells at the last time point. Scale bars for left figures, 50 μm. Scale bars for right figures, 10 μm. e) Migration trajectories of cells showing different nuclear deformation patterns on nanopillars (Ring: N=35 cells; Line: N=42 cells). f) MSD measurement of cells showing different nuclear deformation patterns. g) Comparison of migration rate of cells showing varying nuclear deformation patterns. h) Migration trajectories of MCF-7 cells showing different nuclear deformation patterns on nanopillars (MCF7 ring: N=28 cells; MCF7 line: N=12 cells). Two blue circles with the same diameter are centered with the origin to show that MCF-7 cells with line deformation on nanopillars tend to migrate faster than those showing ring deformation. i) MSD measurement of MCF-7 cells showing different nuclear deformation patterns. j) Comparison of migration rate of MCF-7 cells showing varying nuclear deformation patterns. Statistical significance of migration rate measurement under different conditions was evaluated by an unpaired t-test with Welch's correction. ****P<0.0001; **P<0.01

FIG. 9. Evaluating anti-metastatic drug effects via nanopillar-induced subnuclear deformation. a) Characterize response of cancer cells with varying malignancies to anti-metastatic drug treatment using deformation anisotropicity. b) Nuclear deformation patterns and their orientation of MCF-7 cells and MDA-MB-231 cells on nanopillar arrays with or without curcumin treatment. Scale bar, 5 μm. c) Anisotropy measurement of nanopillar-guided nuclear features in MCF-7 cells and MDA-MB-231 cells with or without curcumin treatment (MCF-7: N=61 pillars (DMSO); N=34 pillars (curcumin); MDA-MB-231: N=34 pillars (DMSO); N=52 pillars (curcumin).). d) Fraction of ring deformation and line deformation in MDA-MB-231 cells and MCF-7 cells on nanopillar arrays with or without curcumin treatment (MCF-7: N=35 cells (DMSO); N=21 cells (curcumin); MDA-MB-231: N=27 cells (DMSO); N=30 cells (curcumin).). Error bars represent SD. e) Wound healing assay of MCF-7 cells and MDA-MB-231 cells with or without curcumin treatment for 24 h. Scale bars, 200 μm. f) Migration rate of MCF-7 cells and MDA-MB-231 cells under different conditions was measured using wound healing assay (N=3 batches). Statistical significance of measurement for coherency and migration rate under different conditions was evaluated by an unpaired t-test with Welch's correction. ****P<0.0001; *P<0.05; ns>0.05.

FIG. 10. Raw images of nuclear morphology of MDA-MB-231 cells and MCF-7 cells on flat versus on nanopillar arrays. a) Nuclear morphology of MCF-7 cells on flat surfaces and nanopillar-guided nuclear features in MCF-7 cells. b) Nuclear morphology of MDA-MB-231 cells on flat surfaces and nanopillar-induced nuclear shape irregularities in MDA-MB-231 cells. Scale bars, 5 μm.

FIG. 11. Nuclear morphology of MCF-7 cells and MDA-MB-231 cells on flat surfaces and nanopillar arrays. Orientation analysis of nuclear shape irregularities and nanopillar-guided nuclear features. Random orientation of nuclear shape irregularities in MDA-MB-231 cells on flat surfaces. Highly oriented features in MDA-MB-231 cells and poorly oriented features in MCF-7 cells are observed on nanopillar arrays.

FIG. 12. Deformation anisotropicity reveals malignancy in liver cancer. a) Wound healing assay revealed liver cancer cell lines with varying motility. Scale bar, 100 μm. b) Nanopillar-guided nuclear features in PLC-PRF-5 and SK-HEP-1 cells. Scale bar, 5 μm. c) Anisotropy of nanopillar-guided nuclear features in different liver cancer cells. d) Fraction of ring deformation and line deformation in different liver cancer cells. Statistical significance of measurement for coherency under different conditions was evaluated by an unpaired t-test with Welch's correction. ****P<0.0001.

FIG. 13. MCF-7 cells with (a) ring and (b) line deformation on nanopillars. Scale bars, 5 μm.

FIG. 14. Deformation anisotropicity is dependent with anti-metastatic drug concentration. a) Nanopillar-guided nuclear features in MDA-MB-231 cells with DMSO, 1 μM and 10 μM curcumin treatment. Scale bar, 5 μm. b) Anisotropy of nanopillar-guided nuclear features reveals the anti-metastatic drug effect with different drug concentrations. c) Fraction of ring deformation and line deformation in MDA-MB-231 cells with DMSO, 1 μM and 10 μM curcumin treatment. Statistical significance of measurement for coherency under different conditions was evaluated by an unpaired t-test with Welch's correction. ****P<0.0001;**P<0.01; *P<0.05.

FIG. 15. Nanopillar-guided nuclear deformation enables anti-metastatic drug screening. a) Nanopillar-guided nuclear features in MDA-MB-231 cells with or without 10 μM haloperidol treatment. Scale bar, 5 b) Anisotropy of nanopillar-guided nuclear features reveals the anti-metastatic drug effect. c) Fraction of ring deformation and line deformation in MDA-MB-231 cells with or without 10 μM haloperidol treatment. Statistical significance of measurement for coherency under different conditions was evaluated by an unpaired t-test with Welch's correction. **P<0.01.

FIG. 16. Degree of subnuclear shape irregularities is dependent with lamin A level in SK-N-SH cells a) Fluorescent images showing that cells with high lamin A level show abnormal nuclear morphology. Scale bar, 20 μm. b) Characterization of whole-nucleus morphology between low-lamin A cells and high-lamin A cells. c) Raw images showing high lamin A cells exhibit abnormal nuclear morphology while low lamin A cells display a regular nuclear contour. Scale bar, 5 μm. d) Schematics showing differential nanopillar-guided features in low- and high-lamin A cells. e) SEM of nanopillar arrays and fluorescent images showing that subnuclear grooves have been guided by nanopillars in high-lamin A cells, whereas ring-like features were observed in low-lamin A cells. Scale bars, 2 μm (SEM). 10 μm (BF). f) Anisotropy measurement of the nanopillar-guided subnuclear features in low-lamin A cells (N=122 pillars) and high-lamin A cells (N=48 pillars). g) Fraction of ring deformation and line deformation in low-lamin A cells (N=64 cells) and high-lamin A cells (N=24 cells). Statistical significance was evaluated by an unpaired t-test with Welch's correction. ****P<0.0001; ns>0.05.

FIG. 17. Elevated lamin A/C level leads to decreased subnuclear irregularities and reduced cell motility a) Fluorescent images showing ring deformation patterns in SK-N-SH cells transfected with lamin A/C-EGFP. Scale bar, 5 μm. b) Anisotropy measurement of the nanopillar-guided subnuclear features in both untransfected and transfected cells. c) Live cell imaging showing overnight migration of transfected and untransfected cells and fluorescent images showing lamin A and lamin A/C-EGFP signals in cells at the last point of cell migration. Green arrow indicates the transfected cells whereas the red arrow indicates the untransfected cell. Scale bars, 50 μm. d) Correlation between migration rate and lamin A level at single cell level for both untransfected and transfected cells. e) Comparison of migration rate between untransfected cells, including low and high lamin A cells, and transfected cells. Statistical significance was evaluated by an unpaired t-test with Welch's correction. ****P<0.0001; **P<0.01; *P<0.05.

FIG. 18. Subnuclear irregularities and lamin A level correlates with EMT a) Fluorescent images showing that cells with high lamin A show strong vimentin signal. Scale bar, 10 μm. b) Correlation between lamin A level and vimentin level in individual SK-N-SH cells. c) High-lamin A cells (N=48 cells) have a higher vimentin intensity compared with low-lamin A cells (N=141 cells). d) Fluorescent images showing that cells with guided nuclear grooves show stronger vimentin signal than those exhibiting subnuclear ring features on nanopillar arrays. Scale bar, 10 μm. e) Correlation between anisotropicity of guided nuclear irregularities and vimentin level in individual SK-N-SH cells. f) Cells showing subnuclear ring patterns (N=22 cells) have a significantly lower vimentin intensity compared with those with subnuclear line patterns (N=16 cells). Statistical significance was evaluated by an unpaired t-test with Welch's correction. ****P<0.0001.

FIG. 19. Probe anti-cancer drug effect on neuroblastoma cells via subnuclear anisotropicity on nanopillars. a) Fluorescent images showing that anti-cancer drug treatment leads to increased lamin A level in SK-N-SH cells. Scale bar, 100 μm. b) Lamin A intensity measurement revealed that cells treated with anti-cancer drugs have a significantly higher lamin A level than those treated with DMSO. c) Nanopillar-guided subnuclear grooves become less upon anti-cancer drug treatment. Scale bars, 5 μm. d) Anisotropy measurement of the nanopillar-guided subnuclear features in cells treated with DMSO, 1 μM DOX and 10 μM Etop. e) Fraction of ring deformation and line deformation in cells treated with DMSO, 1 μM DOX and 10 μM Etop. Statistical significance was evaluated by an unpaired t-test with Welch's correction. ***P<0.001; *P<0.05.

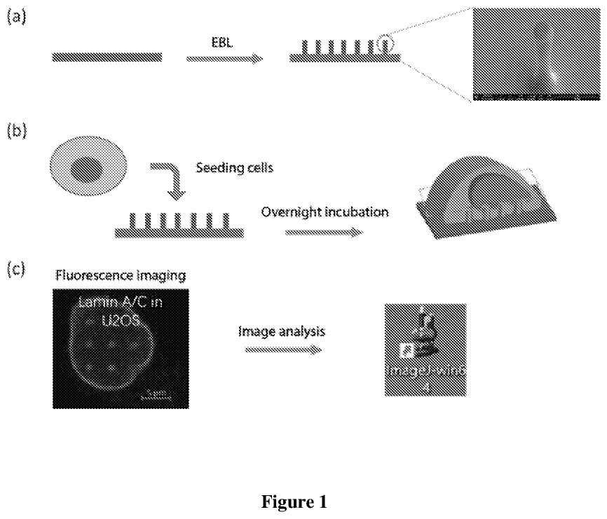

### DETAILED DESCRIPTION

The specification teaches a method of grading the morphology of a tumor cell.

Disclosed herein is a method of grading the morphology of a tumor cell, the method comprising:
a) culturing the tumor cell on an array comprising a plurality of nanopillars; and
b) assessing a deformation pattern of the nucleus to provide an indication of the morphology of the tumor cell.

In one embodiment, the morphology is a nuclear morphology.

In one embodiment, the array comprising a plurality of nanopillars is capable of inducing nanometer scale deformation pattern in the nucleus of the tumor cell.

Without being bound by theory, the present invention is the first one of this kind to combine vertically aligned nanostructures with nuclear abnormality grading. Different from the current clinical practice which is heavily based on human inspection of nuclear abnormalities, the invention provides a novel platform to guide the abnormal nuclear features into detectable patterns for objective and quantitative evaluation in tumor grading. It is also significantly different from the polymer-based nano-micropillar arrays which only induce global deformation per nucleus at the micrometer scale, but neglecting the clinical relevant nanoscale deformations. Although inorganic nanopillars have been recently reported to measure the nuclear stiffness in cells, the previous work was solely based on the depth of nucleus deformation instead of the characterization on the nanoscale morphological pattern reorganization. In addition, no study on the correlation between nuclear deformation and cancer malignancy or tumor grading was conducted using nanopillar previously, as only 3T3 or primary neurons were used instead of cancer cells with different malignancy status.

The term “nanometer scale deformation pattern” may refer to a deformation pattern that is in the scale of, for example, less than 1 μm, between 100 nm to less than 1 μm, between 10 nm to less than 100 nm or between 1 nm to less than 10 nm. The deformation pattern may be less than 900 nm, less than 800 nm, less than 700 nm, less than 600 nm, less than 500 nm, less than 400 nm, less than 300 nm, less than 200 nm, less than 100 nm, less than 90 nm, less than 80 nm, less than 70 nm, less than 60 nm, less than 50 nm, less than 40 nm, less than 30 nm, less than 20 nm, or less than 10 nm.

The term “tumor,” as used herein, refers to any neoplastic cell growth and proliferation, whether malignant or benign, and all pre-cancerous and cancerous cells and tissues. The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. The term “precancerous” refers to a condition or a growth that typically precedes or develops into a cancer. By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer. By “early stage cancer” is meant a cancer that is not invasive or metastatic or is classified as a Stage 0, I, or II cancer. The term “late stage cancer” generally refers to a Stage III or Stage IV cancer, but can also refer to a Stage II cancer or a substage of a Stage II cancer. One skilled in the art will appreciate that the classification of a Stage II cancer as either an early stage cancer or a late stage cancer depends on the particular type of cancer. Illustrative examples of cancer include, but are not limited to, breast cancer, prostate cancer, ovarian cancer, cervical cancer, pancreatic cancer, colorectal cancer, lung cancer, hepatocellular cancer, gastric cancer, liver cancer, bladder cancer, cancer of the urinary tract, thyroid cancer, renal cancer, carcinoma, melanoma, brain cancer, non-small cell lung cancer, squamous cell cancer of the head and neck, endometrial cancer, multiple myeloma, rectal cancer, and esophageal cancer.

Particular types of tumors include hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, squamous cell carcinoma, basal cell carcinoma, adenocarcinoma (well differentiated, moderately differentiated, poorly differentiated or undifferentiated), renal cell carcinoma, hypernephroma, hypernephroid adenocarcinoma, bile duct carcinoma, choriocarcinoma, seminoma, embryonal carcinoma, Wilms' tumor, testicular tumor, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, and non-Hodgkin's lymphoma.

In one embodiment, the method characterizes a cell as an early stage cancer (such as Stage 0, I or II cancer) or a late stage cancer (such as Stage III or IV).

The most common forms of cancer arise in somatic cells and are predominantly of epithelial origin, e.g., prostate, breast, colon, urothelial and skin, followed by cancers originating from the hematopoetic lineage, e.g., leukemia and lymphoma, neuroectoderm, e.g., malignant gliomas, and soft tissue tumors, e.g., sarcomas.

Malignant transformation represents the transition to a malignant phenotype based on irreversible genetic alterations. Although this has not been formally proven, malignant transformation is believed to take place in one cell, from which a subsequently developed tumor originates (the “clonality of cancer” dogma). Carcinogenesis is the process by which cancer is generated and is generally accepted to include multiple events that ultimately lead to growth of a malignant tumor. This multi-step process includes several rate-limiting steps, such as addition of mutations and possibly also epigenetic events, leading to formation of cancer following stages of precancerous proliferation. The stepwise changes involve accumulation of errors (mutations) in vital regulatory pathways that determine cell division, asocial behavior and cell death. Each of these changes may provide a selective Darwinian growth advantage compared to surrounding cells, resulting in a net growth of the tumor cell population. A malignant tumor does not only necessarily consist of the transformed tumor cells themselves but also surrounding normal cells which act as a supportive stroma. This recruited cancer stroma consists of connective tissue, blood vessels and various other normal cells, e.g., inflammatory cells, which act in concert to supply the transformed tumor cells with signals necessary for continued tumor growth.

In current cancer pathology practice, the nuclear abnormality has to be inspected and assessed by individual pathologies under a microscope. The grading system is fairly descriptive and hard to be quantified objectively. For instance, there are four grades classified in the WHO/International Society of Urologic Pathology (ISUP) grading system, which are defined as Grade 1, nucleoli absent or inconspicuous and basophilic at 40× (rare); Grade 2, nucleoli not prominent at 10× but visible and eosinophilic at 40× (40% of tumors); Grade 3, nucleoli conspicuous and eosinophilic at 10× (30-40% of tumors); and Grade 4, extreme nuclear pleomorphism, multinucleated cells, rhabdoid or sarcomatoid differentiation (15% of tumors). Clearly, the judgment of the actual biopsy sample is highly subjective and dependent on the experience of individual pathologies which is inevitably objective and subject to compromised accuracy and reproducibility.

In one embodiment, the method characterizes a cell as Grade 1, Grade 2, Grade 3 or Grade 4 according to the ISUP system.

The method as defined herein may, for example, be used to grade a prostate cancer, a renal cancer or a bladder cancer.

Malignant tumors can also be categorized into several stages according to classification schemes specific for each cancer type. The most common classification system for solid tumors is the tumor-node-metastasis (TNM) staging system. The T stage describes the local extent of the primary tumor, i.e., how far the tumor has invaded and imposed growth into surrounding tissues, whereas the N stage and M stage describe how the tumor has developed metastases, with the N stage describing spread of tumor to lymph nodes and the M stage describing growth of tumor in other distant organs. Early stages include: T0-1, N0, M0, representing localized tumors with negative lymph nodes. More advanced stages include: T2-4, N0, M0, localized tumors with more widespread growth and T1-4, N1-3, M0, tumors that have metastasized to lymph nodes and T1-4, N1-3, M1, tumors with a metastasis detected in a distant organ. Staging of tumors is often based on several forms of examination, including surgical, radiological and histopathological analyses. In addition to staging, there is also a classification system to grade the level of malignancy for most tumor types. The grading systems rely on morphological assessment of a tumor tissue sample and are based on the microscopic features found in a given tumor. These grading systems may be based on the degree of differentiation, proliferation and atypical appearance of the tumor cells. Examples of generally employed grading systems include Gleason grading for prostatic carcinomas and the Nottingham Histological Grade (NHG) grading for breast carcinomas.

In one embodiment, the method characterizes a cell as under the T stage, N stage or M stage under the tumor-node-metastasis (TNM) staging system.

In one embodiment, the method enables quantitative measurement for objective grading and minimized human bias. The present invention may allow abnormal nuclear features to be grated into ordered patterns for easy recognition and objective quantification.

In one embodiment, the tumor is a cancer. In one embodiment, there is provided a method of grading the morphology of a cancer cell, the method comprising:
a) culturing the cancer cell on an array comprising a plurality of nanopillars; and
b) assessing a deformation pattern of the nucleus to provide an indication of the morphology of the cancer cell.

The method as defined herein may be combined with other molecular-biology techniques such as immunofluorescence, DNA or RNA sequencing techniques to obtain more information from a tumor or cancer cell. For example, the immunofluorescence technique may be used to detect protein expression patterns (such as tumor biomarkers) or can be used to detect nuclear proteins (such as a Lamin protein) to allow the nucleus to be visualized.

In one embodiment, step b) comprises staining and visualising the nucleus by microscopy. The method may comprise probing the nucleus with an antibody or other techniques that are well known in the art, such as nucleic acid hybridization techniques (e.g. fluorescence in-situ hybridization).

The method may comprise comparing the morphology (or nuclear morphology) of the tumor cell with a reference. The reference may, for example, be from a normal or healthy cell or a tumor cell that has previously been characterized.

In one embodiment, the method distinguishes the cancer cell from a non-cancer cell (such as a benign, normal or healthy cell).

In one embodiment, the method assesses the malignancy potential of the cancer or tumor cell. An isotropic deformation pattern may indicate low-malignancy of the tumor or cancer cell. A linear or anisotropic deformation pattern may indicate high-malignancy of the tumor or cancer cell. In one embodiment, the method distinguishes a cancer or tumor cell between one that is highly-malignant and one that is lowly malignant.

The term “isotropic deformation pattern” may refer to a deformation pattern that is evenly distributed when measured from different directions.

The term “linear deformation pattern” or “anisotropic deformation pattern” may refer to a deformation pattern that is not evenly distributed when measured from different directions.

In one embodiment, the method evaluates the probability or likelihood of tumor cells of becoming metastatic. The probability or likelihood, may for example, be a 10%, 20%, 30%, 40%, 50%, 60%, 70%, 80%, 90% or 100% chance of becoming metastatic.

The method may comprise assessing the metastatic potential of a cancer cell. An isotropic deformation pattern may indicate a non-metastatic tumor or cancer cell. A linear or anisotropic deformation pattern may indicate a metastatic tumor or cancer cell. In one embodiment, the method distinguishes a cancer or tumor cell between one that is non-metastatic and one that is metastatic.

The method may comprise detecting a deformation pattern using microscopy such as confocal microscopy.

In one embodiment, the tumor cell is obtained from a subject. The subject may be one who is suffering or suspected of suffering from cancer.

As used herein, the term “subject” includes any human or non-human animal. In one embodiment, the subject is a human. The term “non-human animal” includes all vertebrates, e.g., mammals and non-mammals, such as non-human primates, sheep, dog, cow, chickens, amphibians, reptiles, etc.

The tumor cell as referred to herein may be contained in a sample. A sample can be a biological sample which refers to the fact that it is derived or obtained from a living organism. A “biological sample” also refers to a cell or population of cells or a quantity of tissue or fluid from a subject. Most often, a sample has been removed from a subject, but the term “biological sample” can also refer to cells or tissue analyzed in vivo, i.e., without removal from the subject. The biological sample may be from a resection, bronchoscopic biopsy, or core needle biopsy of a primary, secondary or metastatic tumor, or a cellblock from pleural fluid. In addition, fine needle aspirate biological samples are also useful. In one embodiment, a biological sample is ascites. Biological samples also include explants and primary and/or transformed cell cultures derived from patient tissues. A biological sample can be provided by removing a sample of cells from subject, but can also be accomplished by using previously isolated cells or cellular extracts (e.g. isolated by another person, at another time, and/or for another purpose). Archival tissues, such as those having treatment or outcome history may also be used. Biological samples include, but are not limited to, tissue biopsies, scrapes (e.g. buccal scrapes), whole blood, plasma, serum, urine, saliva, cell culture, or cerebrospinal fluid. The biological sample may be a solid biopsy sample or a liquid biopsy sample. The liquid biopsy sample may be one that contains circulating tumor cells.

In one embodiment, there is provided an array comprising a plurality of nanopillars. The nanopillars may be vertically aligned.

The nanopillars may each have a diameter between about 0.1 micron to about 2 microns, between about 0.1 micron to about 1.5 micron, between about 0.1 micron to about 1.0 micron, between about 0.1 micron to about 0.9 micron, between about 0.2 micron to about 0.8 micron, between about 0.3 micron to about 0.7 micron, between about 0.4 micron to about 0.6 micron, or about 0.5 micron. The nanopillars may each have a diameter of about 0.1 micron, about 0.2 micron, about 0.3 micron, about 0.4 micron, about 0.5 micron, about 0.6 micron, about 0.7 micron, about 0.8 micron, about 0.9 micron, about 1.0 micron, about 1.1 micron, about 1.2 micron, about 1.3 micron, about 1.4 micron, about 1.5 micron, about 1.6 micron, about 1.7 micron, about 1.8 micron, about 1.9 micron or about 2.0 micron. In one embodiment, the diameter is about 0.5 micron.

The nanopillars may be positioned at regular intervals from one another. The nanopillars may have a pitch of between about 0.5 microns and about 8 microns, between about 1 microns and about 5 microns, between about 2 microns and about 4 microns, or about 3 microns In one embodiment, the nanopillars have a pitch of between about 2.0 microns, about 2.1 microns, about 2.2 microns, about 2.3 microns, about 2.4 microns, about 2.5 microns, about 2.6 microns, about 2.7 microns, about 2.8 microns, about 2.9 microns, about 3.0 microns, about 3.1 microns, about 3.2 microns, about 3.3 microns, about 3.4 microns, about 3.5 microns, about 3.6 microns, about 3.7 microns, about 3.8 microns, about 3.9 microns, about 4.0 microns, about 4.1 microns, about 4.2 microns, about 4.3 microns, about 4.4 microns, about 4.5 microns, about 4.6 microns, about 4.7 microns, about 4.8 microns, about 4.9 microns or about 5.0 microns. In one embodiment, the nanopillars have a pitch of about 3.0 microns.

The nanopillars may have a height of between about 0.5 microns to about 20 microns. The nanopillars may have a height of about 0.5 microns, about 0.6 microns, about 0.7 microns, about 0.8 microns, about 0.9 microns, about 1.0 microns, about 1.1 microns, about 1.2 microns, about 1.3 microns, about 1.4 microns, about 1.5 microns, about 1.6 microns, about 1.7 microns, about 1.8 microns, about 1.9 microns, about 2.0 microns, about 2.5 microns, about 3 microns, about 3.5 microns, about 4 microns, about 4.5 microns, about 5 microns, about 5.5 microns, about 6 microns, about 6.5 microns, about 7 microns, about 7.5 microns, about 8.0 microns, about 8.5 microns, about 9.0 microns, about 9.5 microns, about 10 microns, about 10.5 microns, about 11 microns, about 11.5 microns, about 12 microns, about 12.5 microns, about 13 microns, about 13.5 microns, about 14 microns, about 14.5 microns, about 15 microns, about 15.5 microns, about 16 microns, about 16.5 microns, about 17 microns, about 17.5 microns, about 18 microns, about 18.5 microns, about 19 microns, about 19.5 microns or about 20 microns. In one embodiment, the nanopillars have a height of about 1.5 microns.

The nanopillars may also be referred to as nanorods, nanotubes or nanostructures.

The array may be coated with a biomolecule. The biomolecule may be a polymer. These molecules are well known to those of ordinary skilled in the art and comprise antigens, antibodies, cell adhesion molecules such as cadherin or fragment thereof, extracellular matrix molecules such as laminin, fibronectin, vitronectin, collagen, synthetic peptides, carbohydrates and the like. In one embodiment, the array is coated with a polymer. The polymer may be fibronectin, poly-L-lysin (PLL), poly-D-lysin (PDL), gelatin, collagen or any other polymer that is well known in the art. In one embodiment, the array is coated with fibronectin.

The present invention may allow deformation patterns to be easily screened and can be coupled with computational tools for image analysis, and therefore is compatible with automatic processing for high-throughput screening of pathological samples. This can enable simultaneous objective quantitative analysis of multiple nuclei that can be scaled-up to execute high-throughput screening for nanoscale nuclear abnormalities for large sample sizes.

Disclosed herein is a method of grading a sample of tumor cells, comprising:
a) obtaining an image of one or more cells of the sample captured on an array of nanopillars; and
b) determining a nanopillar-induced deformation profile of the one or more cells.

The determination of the deformation profile (or pattern) may comprise determining an angular distribution of orientations in a plurality of regions of interest (ROIs) of the image. In one embodiment, the determination of the deformation profile comprises determining an angular distribution of orientations in a plurality of regions of interest (ROIs) of the image, each ROI containing a nanopillar.

In one embodiment, the method comprises determining a coherency in each ROI.

The coherency may range from a value of 0 to 1, with 0 representing a completely isotropic pattern (e.g. a perfect circle) while 1 refers to an extremely anisotropic pattern (i.e. a straight line). A low-malignant cell line will, for example, have a lower coherency value than a high malignant cell.

In one embodiment, the coherency may be compared to a reference (or threshold value). A coherency that is higher than the reference would indicate that the cell is a high-malignant cell, whereas a coherency of less than the reference would indicate that the cell is a low-malignant cell. In one embodiment, the reference is about 0.2, about 0.3, about 0.4, about 0.5 or about 0.6. In one embodiment, the reference is about 0.3.

In one embodiment, the deformation profile or pattern comprises one or more quantifiable morphological parameters. The quantifiable morphological parameters may be at the nanoscale level. The one or more quantifiable morphological parameters may, for example, be area, length or aspect ratio of a particular region of a nucleus (e.g. a sub-nuclear feature) or an ROI of the image. The particular region may contain a sub-nuclear feature such as a ring, line, connecting line or a patch. A change (e.g. an increase or decrease) of these quantifiable morphological parameters as compared to a reference (such as a cell sample from a healthy subject) may indicate the degree of nuclear deformation.

Disclosed herein is a microfluidic chip for performing a method as defined herein. The microfluidic chip may comprise one or more microfluidic channels (e.g. 2, 3, 4, 5, 6, 7, 8, 9, 10 or more microfluidic channels). The use of microfluidics in the methods as described herein significantly reduces the amount of sample needed for detection. The microfluidic channels can have multiple functions. Each channel can be fluidically independent (e.g. having its own fluid inlet and outlet). The microfluidic channels can be used to isolate or separate cells (e.g. tumor or cancer cells) from a sample. The microfluidic channels can be used to facilitate mixing of the sample with a probe to visualize the cell and/or membrane. These steps may be performed concurrently or sequentially. The microfluidic chip may also comprise one or more chambers having a surface or array comprising a plurality of nanopillars. This may allow culturing of one or more cells on the surface or array, thus allowing one to assess a deformation pattern of the nucleus of the one or more cells.

In one embodiment, the methods as defined herein may be used to probe the heterogeneity of tumor cells. The methods may be combined with other techniques to characterize a panel of proteins, genes or other biomarkers such various techniques (including cell-based, nucleic acid-based, protein-based, metabolite-based, and lipid-based techniques). The methods may be performed at a single-cell level, such as in a microfluidic chip. The methods may be used for cancer diagnosis or to monitor a patient's response to cancer therapy or predict cancer recurrence.

Disclosed herein is a method of predicting the progression of a tumour in a subject, the method comprising:
a) culturing a cell sample on a surface comprising a plurality of nanopillars; and
b) assessing the degree of nuclear deformation of cells of the cell sample to provide an indication of the progression of the tumor in the subject.

In one embodiment, there is provided a method of predicting the progression of a cancer in a subject, the method comprising:
a) culturing a cell sample on a surface comprising a plurality of nanopillars; and
b) assessing the degree of nuclear deformation of cells of the cell sample to provide an indication of the progression of the cancer in the subject.

In one embodiment, the method comprises obtaining a cell sample from the subject prior to step a).

In one embodiment, there is provided a method of predicting the prognosis of a tumor in a subject, the method comprising:
a) culturing a cell sample on a surface comprising a plurality of nanopillars; and
b) assessing the degree of nuclear deformation of cells of the cell sample to predict the prognosis of the tumor in the subject.

In one embodiment, the degree of nuclear deformation may refer to the change in the pattern of nuclear deformation of cells of the cell sample as compared to a reference. The reference may be a cell sample from a healthy or control subject.

The term “prognosis” as referred to herein refers to a prediction of the probable course and outcome of a clinical condition or disease. A prognosis of a patient is usually made by evaluating factors or symptoms of a disease that are indicative of a favorable or unfavorable course or outcome of the disease. The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. A prognosis may be expressed as the amount of time a patient can be expected to survive. Alternatively, a prognosis may refer to the likelihood that the disease goes into remission or to the amount of time the disease can be expected to remain in remission. Prognosis can be expressed in various ways; for example prognosis can be expressed as a percent chance that a patient will survive after one year, five years, ten years or the like. Alternatively prognosis may be expressed as the number of months, on average, that a patient can expect to survive as a result of a condition or disease. The prognosis of a patient may be considered as an expression of relativism, with many factors effecting the ultimate outcome. For example, for patients with certain conditions, prognosis can be appropriately expressed as the likelihood that a condition may be treatable or curable, or the likelihood that a disease will go into remission, whereas for patients with more severe conditions prognosis may be more appropriately expressed as likelihood of survival for a specified period of time.

Disclosed herein is a method of predicting the likelihood of a metastatic cancer in a subject, the method comprising:
a) culturing a cell sample on a surface comprising a plurality of nanopillars; and
b) assessing the degree of nuclear deformation of cells of the cell sample to predict the likelihood of a metastatic cancer in a subject.

Disclosed herein is a method of predicting a response of a tumor to a cancer drug in a subject, the method comprising:
a) culturing a cell sample on a surface comprising a plurality of nanopillars; and b) assessing the degree of nuclear deformation of cells of the cell sample to predict the response of the tumor to the cancer drug in the subject.

In one embodiment, the cell sample is obtained from the subject prior to step a).

In one embodiment, the subject has been administered a cancer drug or the cancer sample is treated with a cancer drug.

In one embodiment, the method comprises treating a subject. The term “treating” as used herein may refer to (1) preventing or delaying the appearance of one or more symptoms of the disorder; (2) inhibiting the development of the disorder or one or more symptoms of the disorder; (3) relieving the disorder, i.e., causing regression of the disorder or at least one or more symptoms of the disorder; and/or (4) causing a decrease in the severity of one or more symptoms of the disorder.

By “about” is meant a quantity, level, value, number, frequency, percentage, dimension, size, amount, weight or length that varies by as much 15, 14, 13, 12, 11, 10, 9, 8, 7, 6, 5, 4, 3, 2 or 1% to a reference quantity, level, value, number, frequency, percentage, dimension, size, amount, weight or length.

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

Certain embodiments of the invention will now be described with reference to the following examples which are intended for the purpose of illustration only and are not intended to limit the scope of the generality hereinbefore described.

### EXAMPLES

### Example 1

### Design and Fabrication of Nanochips.

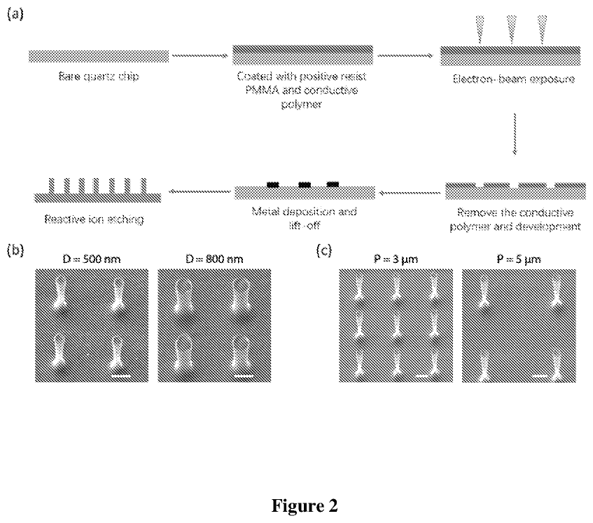

The primary design used for nuclear deformation is nanopillars, which can be fabricated with nanometer controllability using electron-beam lithography (EBL) or other compatible methods with similar geometrical precision. A typical EBL-based fabrication process is shown in FIG. 2a. In brief, a 300 nm layer of electron-sensitive polymer, PMMA A4, and a thin layer of conductive polymer (AR-PC 5090.02) is spin-coated on the quartz substrate. Customized dot patterns with a defined diameter of 200-800 nm and pitch 3 or 5 μm are written by an electron beam and developed in MIBK: IPA=1:3 solution for 45 s to generate an array of holes. Subsequently, a 70 nm layer of chromium is deposited on the surface through thermal evaporation. After the lift-off of the PMMA in acetone, the substrate with a nanopatterned chromium mask can be obtained. Lastly, nanostructures in 1.5 μm height are obtained by reactive ion etching in a mixture of CF₄ and CHF₃. The residual metal mask can be specifically removed by chromium etchant. This nanofabrication process provides good controllability on the dimensional properties of the nanostructures (FIGS. 2b and 2c).

### Generating and Imaging-Guided Nuclear Deformation in Cancer Cells.

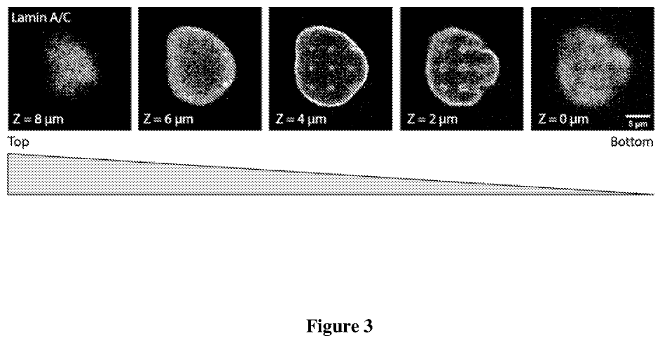

In order to generate quantifiable nuclear deformation patterns on nanopillar chips, the cancer cells will be cultured on the chip and stained with nuclear markers for imaging. Before cell plating, the nanochips will be sterilized by 70% ethanol and then dried and subject to 15 min UV radiation, followed by a 15 min coating of 5 μg/cm² fibronectin to promote cancer cell attachment. After an overnight culture of cancer cells, the nuclear envelope of cells on nanochip will be stained by anti-Lamin A/C or anti-Lamin B1 antibody via immunostaining to visualize the nuclear deformation patterns on nanopillars. The guided nucleus patterns can be imaged by confocal microscopy with 100× objective at a series of z-planes to enable sub-nucleus resolution for feature identification at each nanopillar (FIG. 3).

### Quantification Matrix

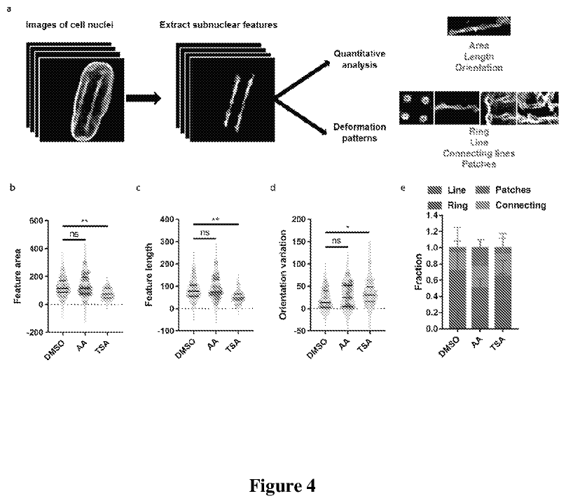

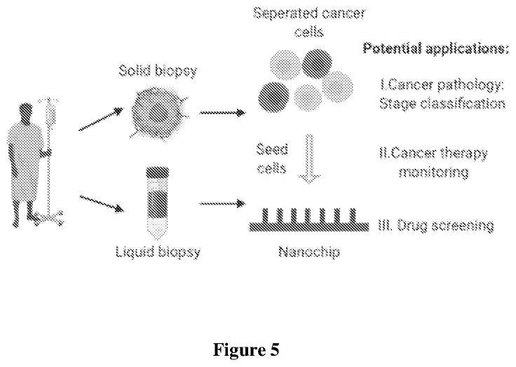

The degree of nuclear irregularities can be assessed by either the quantifiable morphological parameters, like area, length and orientation, or the deformation patterns of the cell nuclei (FIG. 4a, b-d). Different deformation patterns indicate varying degrees of subnuclear abnormalities, for example, more subnuclear ring features refer to fewer nuclear abnormalities, moreover, they are also found to be associated with metastatic potential of cancer cells. The nanopillar-guided subnuclear features have been classified into four groups, ring, line, connecting lines and patches, based on the number of the nuclear grooves guided by the single nanopillar, which represents an increased level of irregularities (FIGS. 4a and e).

### Qualitative Analysis of Nanopillar-Guided Deformation Patterns for Nuclear Grading.

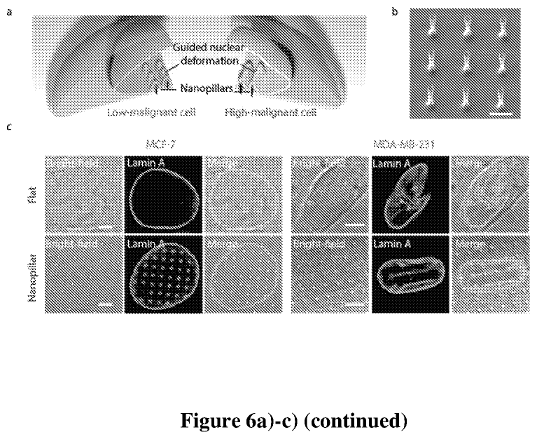

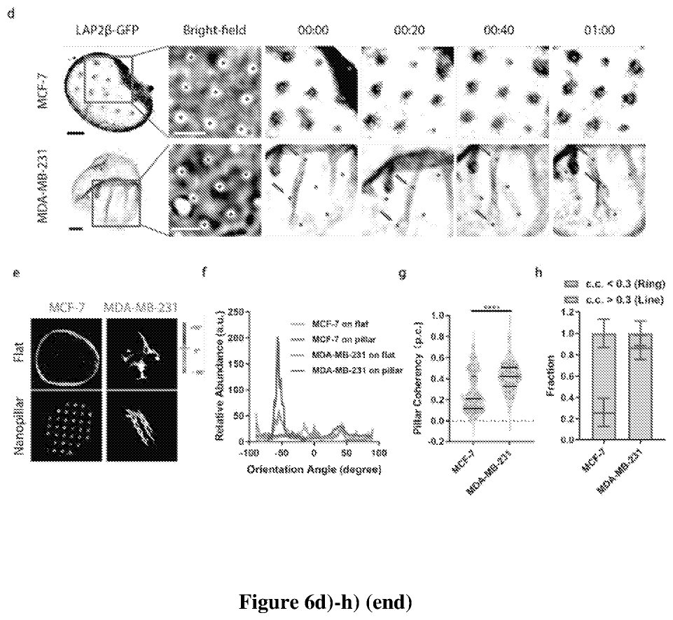

Using nanopillar arrays, nuclei of low malignant cancer cells with less abnormal morphology display isotropic ring patterns, while those of highly malignant cancer cells with significant morphological abnormalities exhibit linear and anisotropic features aligned around nanopillars (FIG. 6). To quantitatively evaluate these nanopillar guided nuclear deformation patterns, the microscopy images will be read and analyzed by an open-source image software, Image J. The directionality of the guided features on nanopillars will be specifically quantified using an Image J plug-in, Orientation J. Two kinds of analysis are performed by orientation J: 1) visualizing the orientation distribution with each color representing one orientation angle in the processed images for direct single-cell inspection; 2) quantifying the orientation coherency of the deformation patterns as a quantitative parameter to characterize different cell types. In the color analysis, around the nanopillar-deformed regions (as indicated with boxes in FIG. 6d), certain dominant colors can be seen in the high-malignant cancer cell representing dominant orientation for the guided pattern; while in the low-malignant cancer cell, no dominant colors are observed for the isotropic features around nanopillars. This analysis enables a color-based visual inspection of individual nuclei (FIG. 6d). For the quantitative analysis, coherency (ranging from 0 to 1) measured at each pillar-deformed region is used as the parameter for nuclear grading with statistical analysis. A higher coherency value indicates a more anisotropic feature. As a proof of concept, breast-cancer cells in high malignancy (MDA-MB-231) showed significantly higher coherency than the low malignant cells (MCF7) with the differential threshold at 0.3 (FIGS. 6g and 6h).

### Digital Cancer Pathology.

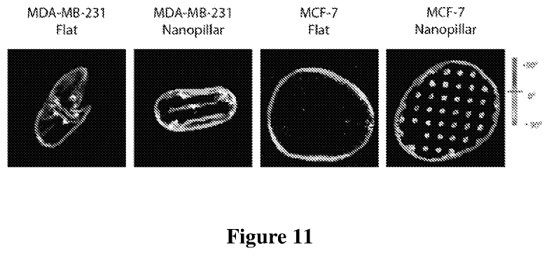

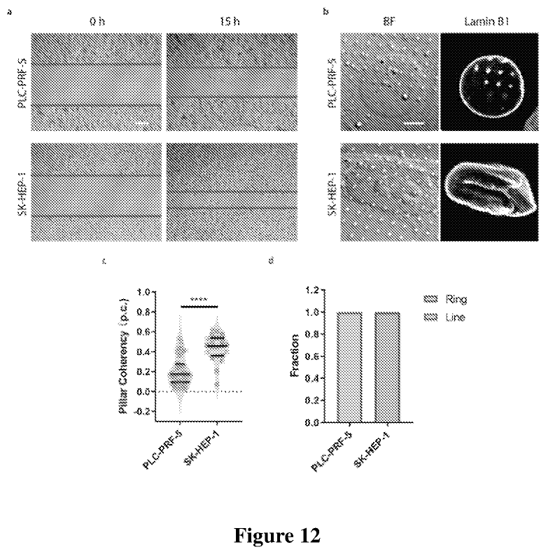

The main bottleneck of current nuclear grading is the human-based inspection and assessment of clinical specimens. With the quantitative indicator of nanoscale nuclear deformation coherency offered by this invention, a digital tumor grading can be developed by combining automatic specimen scanning and computer-aided image processing. Both solid biopsy samples and circulating tumor cells (CTC) enabled liquid biopsy can be examined with a single-cell resolution on nanochips. The guided nuclear abnormality features can be easily located and identified at a designed nanopillar position via automatic image scanning and the coherency values can be analyzed by standardized computer programs Comparing to current human-based practice, the digital cancer pathology will not only enable objective grading with much less human-bias but also greatly speed up the grading throughput with automated scanning and computer-based analysis. The applicability of this method has been tested on a few cancer cell lines, including breast cancer cells demonstrated earlier (FIG. 6) and liver cancer cells. As shown below, PLC-PRF-5 cells, the low-malignant liver cancer cells, show significantly lower coherency compared with SK-HEP-1 cells, the high-malignant liver cancer cells. (FIG. 12).

Probe Cancer Heterogeneity with the Single-Cell Resolution for Cancer Therapy Monitoring.

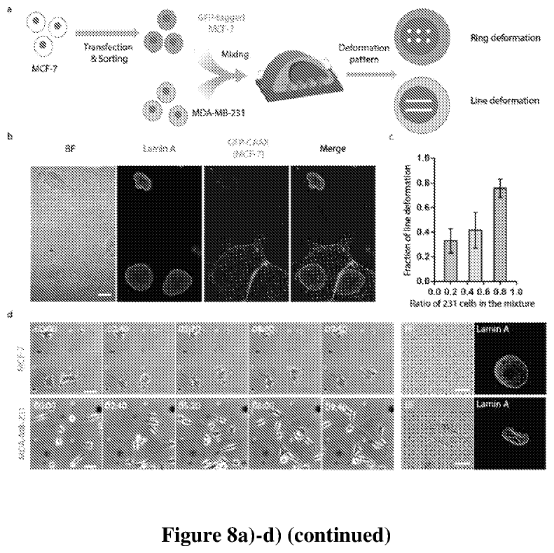

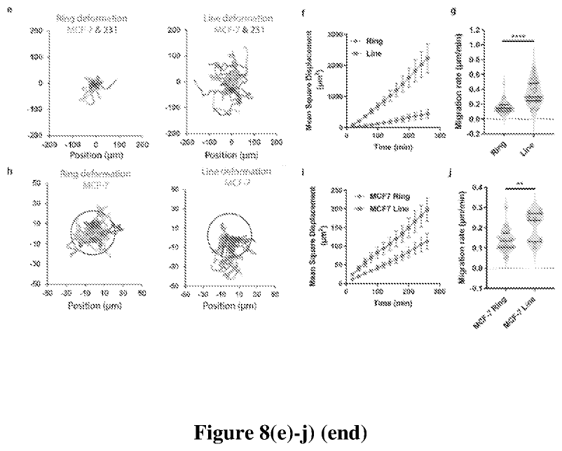

In addition to cancer diagnostics, one may monitor the patient's response to cancer therapy and cancer recurrence. A significant challenge for cancer therapy monitoring is the heterogeneity among individual cells. Even in the same biopsy sample, different cells may have different properties, e.g. malignancy to cause metastasis or resistance to drug treatment. The ability to grade tumor on a single cell resolution is critical in probing the tumor heterogeneity, which gave rise to novel methodologies like Single Cell Analysis (SCA). Various SCA techniques (cell-based, nucleic acid-based, protein-based, metabolite-based, and lipid-based) are presently utilized for cancer heterogeneity characterization. However, most of them require the complex profiling of a panel of proteins, genes or other biomarkers combined with proteomics analysis, gene sequencing, or mass spectrometry for metabolites analysis. Existing biophysical or morphometric methodologies are not efficient in probing tumor heterogeneity. For instance, microfluidic devices can be used for CTC numeration as blood-based liquid biopsies, but the evaluation of cancer heterogeneity often requires down-stream sequencing and other analytical technology for high-resolution single-cell analysis. While AFM can afford to probe the mechanical properties of isolated single cancer cells in high resolution, it is hard to screen a large number of cells. In comparison, using the nanopillar chip described in this invention, single-cell quantification and simultaneous detection of multiple cells can be readily achieved, which will enable the evaluation of the heterogeneity with high-throughput. It can greatly facilitate the detailed monitoring of patients' response to cancer therapy and the probability assessment of cancer recurrence for better patient management. The performance of the nanopillar chip was evaluated using mixed breast cancer cell lines to mimic different heterogeneity conditions. Specifically, GFP-tagged MCF-7 cells and non-labeled MDA-MB-231 cells are mixed in different ratios (from 0.3 to 0.8) to mimic the tumors having varying percentages of cancer cells with different malignancies (FIG. 8a). Consistent with previous tests, MCF-7 cells and MDA-MB-231 cells showed distinct nuclear deformation patterns (FIG. 8b). The percentage of cells showing anisotropic nuclear deformation is correlated with the mixing ratio of MDA-MB-231 cells to MCF-7 cells (FIG. 8c).

### Drug Screening.

In addition to cancer diagnosis, the invention may be used for anticancer drug screening. With more than 90 percent of cancer mortality are caused by metastasis, the development of anti-metastatic drugs becomes a new trend, yet there is no anti-metastatic drug on the market. Lacking an effective screening of drug response from cells with different malignancy is one of the big hindrances. Here, the invention offers a novel way to monitor and quantify tumor cell malignancy, which can serve as a direct tool to evaluate the specificity and potency of drug candidates against high and low malignancy cells.

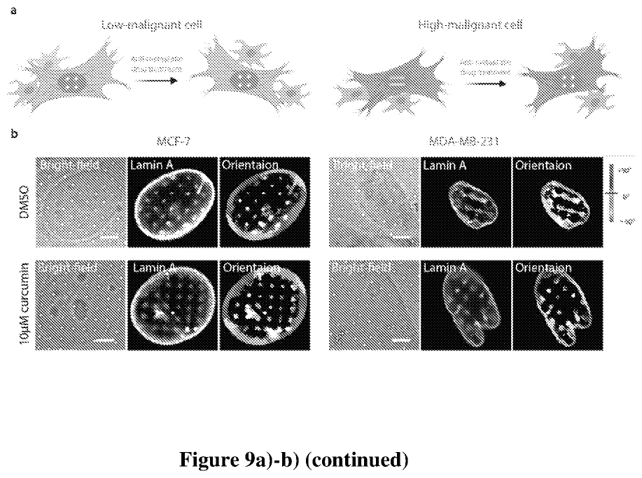

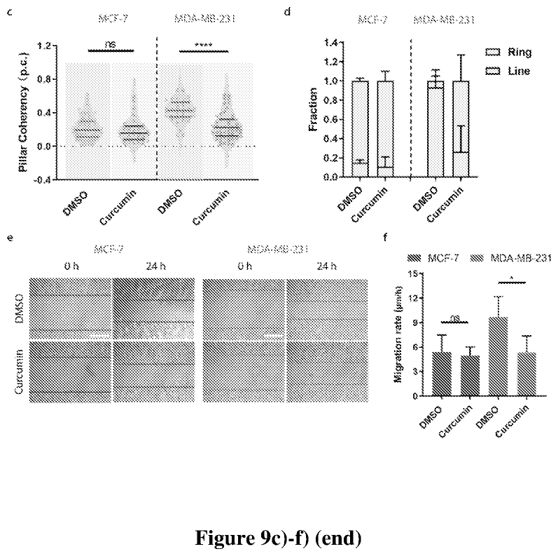

The anti-metastatic drug response of cancer cells with different malignancies can be evaluated. As shown in FIG. 9a-b, when treated by an anticancer drug, curcumin, the high-malignant breast cancer cell, MDA-MB-231, exhibited significant decreases in nuclear deformation coherency on nanopillar arrays. In comparison, low-malignant breast cancer cells, MCF7, showed no significant coherency changes in response to the same drug (FIG. 9a-b).

### Example 2

### Materials and Methods:

### Fabrication and Characterization of Nanopillar Arrays

Nanopillar arrays were fabricated on the quartz chip using electron-beam lithography (EBL) and reactive ion etching (RIE). The quartz chip was cleaned with acetone and isopropyl alcohol and then spin-coated with 300 nm polymethylmethacrylate (PMMA) (MicroChem), followed by coating of one thin conductive layer, AR-PC 5090.02 (Allresist). Designed nanoscale patterns were written on the PMMA layer by electron-beam lithography (FEI Helios NanoLab 650) and the PMMA on the exposed areas was subsequently removed in the 3:1 isopropanol:methylisobutylketone solution. A Cr mask with 80 nm thickness was formed via thermal evaporation (UNIVEX 250 Benchtop), followed by lift-off with acetone. Nanopillars were finally revealed after reactive ion etching with a mixture of CF₄ and CHF₃ (Oxford Plasmalab 80). Characterization of nanopillar dimension was performed using SEM (FEI Helios NanoLab 650) after 10 nm chromium coating.

### Cell Culture and Drug Treatment

Prior to cell culture, the nanostructured chips were cleaned by air plasma for 10 min and exposed to UV for 15 min Subsequently, the nanostructured substrates were coated with fibronectin (2 ug/ml, Sigma-Aldrich) for 30 minutes at 37° C. After coating, cell culture was performed on the substrates. All the cell lines used in this work were maintained in the Dulbecco's Modified Eagle Medium (DMEM) (Gibco) supplemented with 10% fetal bovine serum (FBS) (Life Technologies) and 1% Penicillin-Streptomycin (Life Technologies) in a standard incubator at 37° C. with 5% CO₂. After overnight incubation and the nuclear deformation was stabilized, the MCF-7 and MDA-MB-231 cells on nanostructures are treated with curcumin (Sigma) or DMSO (Sigma). After 24-hour incubation, the treated cells and untreated cells were fixed with 4% Paraformaldehyde (PFA) Solution in PBS (Boster biological technology AR1068) for 15 minutes for subsequent immunostaining

### Immunofluorescence Staining

Cells cultured on nanopillar arrays were immunostained for lamin A or lamin B1. Cells were washed with pre-warmed PBS two times and fixed with 4% paraformaldehyde (PFA) in phosphate-buffered saline (PBS) (Boster biological technology AR1068) for 15 minutes. The cells were washed three times with PBS and then permeabilized with 0.5% Triton X-100 (Sigma) in PBS for 15 minutes. After washing with PBS for three times, samples were blocked using 5% bovine serum albumin (BSA) (Sigma) in PBS for 1 hour before staining with 1:400 anti-lamin A (Abcam ab26300) and anti-lamin B1 (gift from the Saggio lab in Sapienza University of Rome). Samples were washed three times with PBS and stained with the secondary antibody, Chicken anti-Rabbit IgG (H+L) Cross-Adsorbed Secondary Antibody, Alexa Fluor 488 (Invitrogen A21441), 1:500 in staining buffer for 1 hour under room temperature.

### Confocal Imaging and Live Cell Tracking

Imaging of the fluorescently labeled cells on nanopillar arrays was performed using laser scanning confocal microscopy (Zeiss LSM 800 with Airyscan). In particular, a Plan-Apochromat 100×/1.4 oil objective was used. During imaging, fixed cells were maintained in PBS. Z stack images were acquired with 500 nm distance between each frame. Live cell imaging and the subsequent fluorescence imaging was performed using a spinning disc confocal microscope (SDC) that is built around a Nikon Tit inverted microscope equipped with a Yokogawa CSU-W1 confocal spinning head, a Plan-Apo objective (100×1.45-NA), a back-illuminated sCMOS camera (Orca-Fusion; Hamamatsu). Excitation light was provided by 488-nm/150 mW (Vortran) (for GFP), and all image acquisition and processing were controlled by MetaMorph (Molecular Device) software. The migration of individual cells was manually tracked using imageJ, and their migratory behavior was characterized.

### Measurement of Subnuclear Anisotropicity

Anisotropicity of nanopillar-guided subnuclear shape irregularities was measured on nanopillar arrays. Firstly, one image with the best signal-to-background ratio in the stack was selected for subsequent characterization. Square masks (2.75 μm*2.75 μm) centered by the individual nanopillars were manually drawn based on the brightfield channel. Features that were guided by individual nanopillars were cropped using the generated masks. Subsequently, binary images of the guided features were extracted based on the background intensity (2 times of lowest 10% intensity used as a threshold) in the cropped images. Then, the anisotropicity of the subnuclear shape irregularities was measured as coherency, an indicator for anisotropy property, using orientationJ (a plugin function in ImageJ).

### Transfection

For plasmid transfection in cancer cells, 1 μg plasmid was mixed with 1.5 μl Lipofectamine 3000 (Life Technologies) and 2 μl P3000 reagent (Life Technologies) in Opti-MEM (Gibco) and incubated for 20 mins at room temperature. Before the addition of the transfection mixture, cancer cells were starved with the Opti-MEM (Gibco) medium for 30 mins at 37° C. After 4 hours of incubation, the Opti-MEM (Gibco) medium was replaced with regular culture medium and the cells were allowed to recover overnight before cell sorting or live cell imaging.

### Fluorescence-Activated Cell Sorting

Cells were sorted by using the BD FACS Aria II, and gating was done using the BD FACSDiva™ software (Becton, Dickinson Biosciences). Dead cells were excluded from analysis on the basis of FSC/SSC; cell aggregates or small debris were excluded from analysis on the basis of side scatter (measuring cell granularity) and forward scatter (measuring cell size); lastly, GFP positive cells were sorted on the basis of fluorescence intensity.

### Wound Healing Assay

Cells were maintained in 35 mm dishes for each cell line until approximately 90% confluent. Scratch was made in the confluent monolayer of cells with a sterile 200-0 pipette tip, and fresh culture medium was replaced. Brightfield microscopic pictures were taken of the same field at 24 hours. Migration rate was measured by quantifying the closure area within the same time frame using ImageJ.

### Statistical Analysis

Welch's t tests (unpaired, 2 tailed, not assuming equal SD) were used to evaluate the significance. All tests were performed using Prism (GraphPad Software). Data are presented as mean±SEM or mean±SD as stated in the figure captions. All experiments were repeated at least twice, unless otherwise explicitly stated in the figure captions.

### Results and Discussion:

### Nanopillar Guides Nuclear Shape Irregularities in Cancer Cells

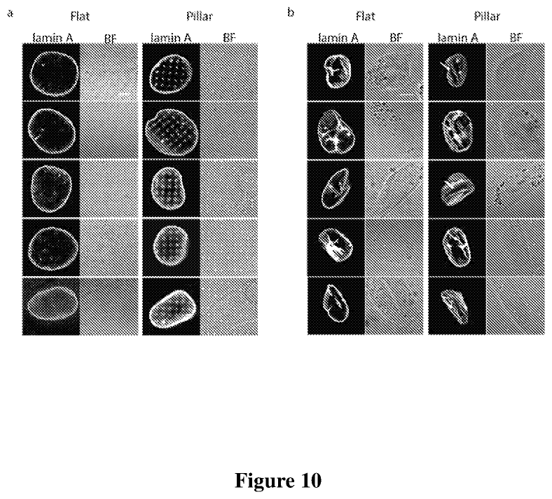

Differential response of subnuclear morphology to nanopillars among tumor cells with different malignancy was first evaluated (FIG. 6a). Arrays of vertically aligned nanopillars with 500 nm diameter, 3 μm pitch, and 1.5 μm height (as shown in the scanning electron micrograph (SEM) (FIG. 6b), were fabricated on transparent quartz coverslip using electron-beam lithography (EBL) and reactive ion etching (RIE). Taking breast cancer cells as a model, low malignant MCF-7 and high malignant MDA-MB-231 cells were examined on both flat and nanopillar arrays. After an overnight culture to allow sufficient generation and stabilization of deformations on nanopillars, the nuclear morphology was visualized via immunostaining of nuclear lamina protein laminA For low malignant MCF7 cells, they display a smooth nuclear outline on flat substrates without obvious subnuclear features (representative image shown in FIG. 6c top left panel and more examples shown in FIG. 10a left column), while those cultured on nanopillar arrays generates an ordered array of rings colocalizing with nanopillar position underneath (FIG. 6c bottom left panel and FIG. 10a right column) In comparison, the high malignant MDA-MB-231 cells show obvious but randomly distributed subnuclear folding and wrinkles across the nucleus (FIG. 6c top right panel and FIG. 10b left column) But when cultured on nanopillar arrays, MDA-MB-231 cells exhibit significantly decreased randomness of subnuclear irregular features. Instead, distinct alignment of subnuclear features into line patterns along adjacent nanopillars are clearly observed (FIG. 6c bottom right panel and FIG. 10b right column). The dynamics of such subnuclear features on nanopillar are further examined in live cells via transient expression of nuclear envelope protein, LAP2β fused with green fluorescent protein (LAP2β-GFP). Strikingly, subnuclear rings in MCF7 cells are relatively stable on each nanopillar location over 1 hour regardless of the overall movement of the whole nucleus (FIG. 6d upper row), while aligned line patterns in MDA-MB-231 cells are able to switch among nearby nanopillar sites following the migration of nucleus (FIG. 6d lower row).

Based on the guided subnuclear features on nanopillars, quantitative analysis is performed to differentiate MCF7 and MDA-MB-231 cells. One pronounced difference is the isotropicity of the laminA patterns: the rings formed in MCF7 nuclei give isotropic distribution around nanopillars; while the lines aligned in MDA-MB-231 cells generate anisotropic intensity profile around nanopillars with dominant distribution in certain angles. To quantitatively distinguish the isotropic and anisotropic patterns of subnuclear deformation on nanopillars, the nuclear boundary was removed and the orientation distribution of the subnuclear lamin A signals was analyzed specifically using the OrientationJ plug-in in ImageJ, where the dominant angle is calculated at each pixel and displayed in color hues. The intact images with nuclear boundaries of example cells in FIG. 6c for each condition are shown in FIG. 11, where the nuclear boundary of MCF7 on flat remains uncut as no subnuclear feature is detectable). As shown in FIG. 6e, the isotropic ring deformation of MCF-7 nuclei on nanopillars exhibits a wide angle distribution of subnuclear features and generates a variety of colors surrounding each nanopillar (FIG. 6e bottom left) Similarly, random distribution of subnuclear groves and invaginations in MDA-MB-231 on flat surfaces and the nuclear boundary of MCF7 without nanopillar guidance also results in a combination of different angles in individual nuclei, and thus a variety of colors (FIG. 6e top left). In contrast, the aligned subnuclear deformation in MDA-MB-231 on nanopillars gives rise to a preferred angle across the whole nucleus and thus a prominent blueish color in display (FIG. 6e bottom left). Collecting the angle distribution inside the individual nucleus, it is obvious that the nanopillar array produce a detectable predominant angle for the anisotropic subnuclear deformation in high malignant MDA-MB-231 cells, while no dominant subnuclear orientation is observed in low malignant MCF7 nucleus (FIG. 6f).

For quantitative evaluation of deformation orientations, the anisotropy of the subnuclear features on each nanopillar is further converted into orientation coherency values (i.e. pillar coherency, or p.c. in short), which is ranging from 0 to 1 with 0 representing a completely isotropic pattern, e.g. a perfect circle and 1 refers to an extremely anisotropic pattern, i.e. straight line. Low-malignant MCF-7 cells have much lower coherency value (p.c.=0.2653±0.181, n=94 pillars) than high-malignant MDA-MB-231 cells (p.c.=0.4165±0.1279, n=44 pillars) (FIG. 6g). A coherency value of 0.3 was set as the threshold to distinguish isotropic and anisotropic subnuclear features in breast cancer cells. By averaging the p.c. values of the same cell, an averaged cell coherency value (in short as c.c.) was further obtained as a single cell readout for cell population analysis. Not surprisingly, MCF-7 sample contains a higher fraction of ring-deformation cells (cell fraction=0.7380±0.1340, n=39 cells) while MDA-MB-231 sample mainly contains line-deformation cells (cell fraction=0.9028±0.1145, n=24 cells), as shown in FIG. 6h. It is interesting to note that both low-malignant MCF-7 cells and high-malignant MDA-MB-231 cells contain a mixed population of low c.c. and high c.c. cells, indicating a heterogeneity even within the same cell type. Similar phenomena were also observed in two liver cancer cells with different migrating speed: fast migrating SK-HEP-1 and slow migrating PLC-PRF-5, as confirmed by wound healing assay (FIG. 12a). When both cell line cultured on nanopillar arrays, that anisotropic line patterns formed in the nuclei of fast migrating SK-HEP-1 cells, whereas slow migrating PLC-PRF-5 cells exhibit isotropic ring patterns on nanopillar arrays (FIG. 12b). The distribution of pillar coherency values shows significant difference between these two cell lines (FIG. 12c), and the cell coherency effectively differentiate them apart (FIG. 12d). These results confirmed that nanopillar arrays can effectively guide subnuclear morphological irregularities in tumor cells and can generate quantifiable subnuclear readouts for cell malignancy evaluation with single cell resolution.

### Nanopillar Geometry Affects the Guidance of Subnuclear Irregularities

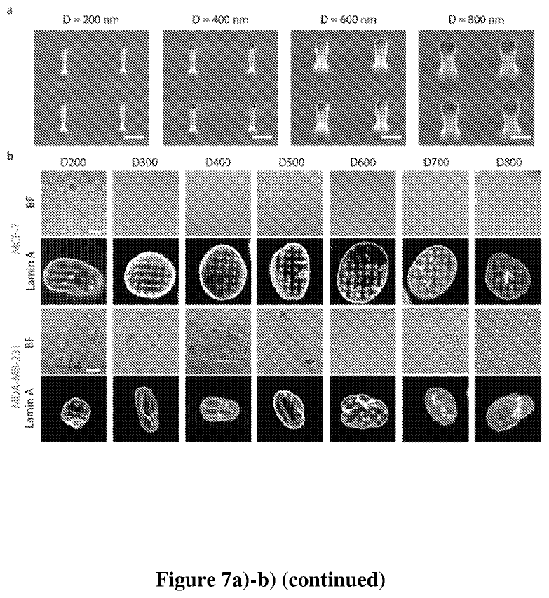

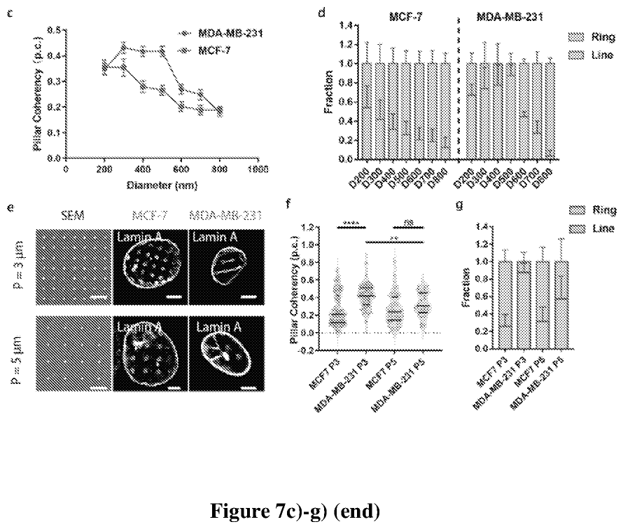

For effective guidance on the subnuclear deformation pattern, a series of nanopillar geometry were surveyed in terms of nanopillar diameter and inter-pillar pitch. Arrays of nanopillars in diameter from 200 nm to 800 nm with 100 nm steps (FIG. 7a) were first fabricated. On pillars with different diameters, MCF-7 cells (FIG. 7b up 2 rows) showed an isotropic ring deformation pattern on large pillars with diameter above 400 nm. However, on smaller pillars with 200 nm and 300 nm diameter, anisotropic line pattern appeared in MCF-7 cells, suggesting a causal correlation between pillar diameter and the nanopillar-induced isotropy of subnuclear features. MDA-MB-231 cells (FIG. 7b bottom 2 rows), in comparison, showed aligned anisotropic line patterns on diameters from 300 to 500 nm, but more isotropic ring features on larger nanopillars of 600 nm to 800 nm, as well as the smallest one (200 nm). By plotting the pillar coherency values of both cell lines across all the diameters tested (FIG. 7c), it was found that nanopillars in 500 nm differentiating the two cell types most effectively. The cell coherency analysis (FIG. 7d) consistently shows that 500 nm gives the largest difference of line deformation between MDA-MB-231 (cell fraction=0.9028±0.1145, n=24 cells) and MCF-7 cell (cell fraction=0.2620±0.1340, n=39 cells). 500 nm for the following experiments was then chosen.

In addition to the diameter effect, the influence of pitch between nanopillars was further examined Nanopillar arrays with center-to-center pitch at 3 μm and 5 μm (SEM with top view in FIG. 7e left) were fabricated for comparison. On nanopillar arrays with 3 μm pitch (FIG. 7e, top row), MCF-7 cells and MDA-MB-231 cells displayed distinguishable ring and line patterns respectively as seen earlier, suggesting an effective pitch for differential guidance on low and high malignant cells. However, on 5 μm pitch arrays (FIG. 7e, bottom row), although the line deformation in MDA-MB-231 cells still falls on each nanopillar, the connection between nearby pillars is reduced. In addition, the number of ring deformation sites per cell in MCF-7 cells also decreased on 5 μm pitch arrays for analysis. Both cases happened likely because the nanopillars are too sparse to provide sufficient guidance on subnuclear features. The impact of pitch between nanopillars are obvious in the analysis of pillar coherency (FIG. 7f) and the fraction of cell in ring and line patterns (FIG. 7g). The pillar coherency of two cell types are significantly different on 3 μm pitch (MCF-7, pc=0.2653±0.1810, n=94 pillars; MDA-MB-231, p.c.=0.4165±0.1279, n=44 pillars; p value<0.0001), but less distinguishable on 5 μm pitch (MCF-7, pc=0.2661±0.1729, n=49 pillars; MDA-MB-231, p.c.=0.3234±0.1377, n=27 pillars; p value=0.1189)) (FIG. 70. Specifically, for MDA-MB-231 cells, changes from 3 μm to 5 μm pitched nanopillar arrays lead to detectable decrease in measured coherency on nanopillars (p value=0.0065) (FIG. 7f). Similar trends are also observed in cell coherency analysis where cell fraction of line patterns shows larger differences on 3 μm pitch arrays (MDA-MB-231: 0.9028±n=24 cells; MCF-7: 0.2620±0.1340, n=39 cells) than 5 μm arrays (MDA-MB-231: mean=0.6799±0.3024, n=27 cells; MCF-7: 0.3150±0.1656, n=38 cells). (FIG. 7g) Arrays with a pitch of 3 μm were chosen for the following experiments.

### Probe Cancer Heterogeneity Via Guided Nuclear Deformation

Taking the advantages of nanopillar-generated multiple sampling points in a single cell nucleus, the ability to quantitatively examine the heterogeneity of malignancy in a given cell population was tested. Heterogeneity among cancer cells presents one of the major hurdles in cancer therapy, as cancer metastasis or drug resistance are often observed only in a subset of cells with higher malignancies. Here a series of cell populations with different heterogeneity was generated by mixing GFP-CAAX tagged low malignant MCF-7 cells with unlabeled high malignant MDA-MB-231 cells in predefined ratios (illustrated in FIG. 8a). A typical microscopy image of a cell mixture containing both cell types on nanopillar arrays is shown in FIG. 8b, where both line deformation in an unlabeled cell and ring deformation in GFP labeled cells were observed. When plotting the fraction of cell with high deformation coherency (>0.3) on nanopillars against the ratio of unlabeled MDA-MB-231 in the cell mixture, a positive correlation between nanopillar-measured fraction and the number of high malignant MDA-MB-231 cells added in the mixture was observed. It indicates that the cancer heterogeneity can be quantitatively characterized via the nanopillar-guided subnuclear deformation pattern (FIG. 8c).

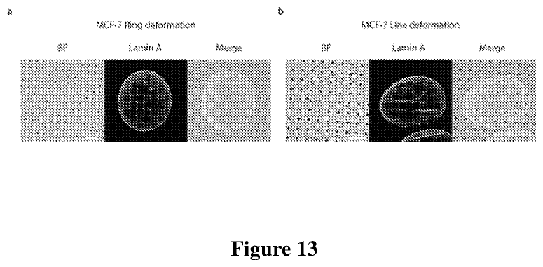

Nanopillar-guided deformation patterns are also strongly correlated with cell migration speed, another key indicator of malignant cells. Cell motility of individual cells obtained from live cell imaging was correlated with their subnuclear deformation patterns on nanopillars for both MCF-7 and MDA-MB-231 cells (FIG. 8d). When pooling together the cells with the same ring or line deformation on nanopillars from both cell types, it was found that cells with ring deformation migrate much slower than line deformation cells despite their cell types, which is clearly shown in cell migration trajectories (FIG. 8e), mean square displacement (MSD) (FIG. 8f), and calculated cell migration rate (FIG. 8g). More interestingly, even among all the MCF-7 cells, two subpopulations with distinct guided nuclear deformation patterns can be differentiated (FIG. 8h-j, and FIG. 13). Consistently, MCF-7 cells with line deformation migrate faster than MCF-7 cells with ring patterns on nanopillars, as similarly measured by longer migration trajectories (FIG. 8h), larger mean square displacement (MSD) (FIG. 8i), and faster cell migration rate (FIG. 8j). Altogether, the nanostructured platform constitutes an effective technology for probing the heterogeneity in a cancer cell population with single cell resolution.

### Evaluate Anti-Metastatic Drug Effect Via Guided Nuclear Deformation

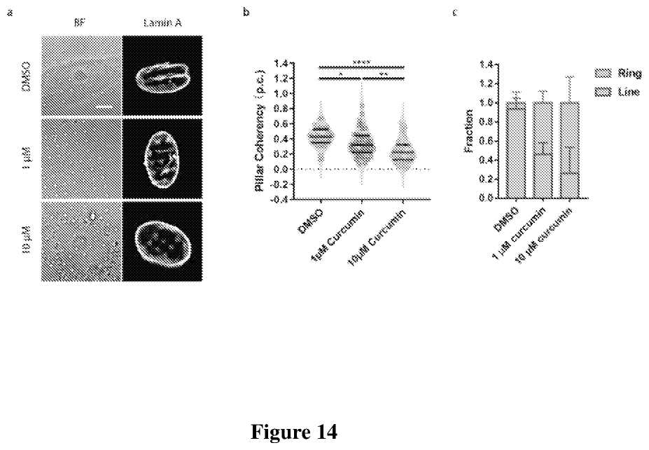

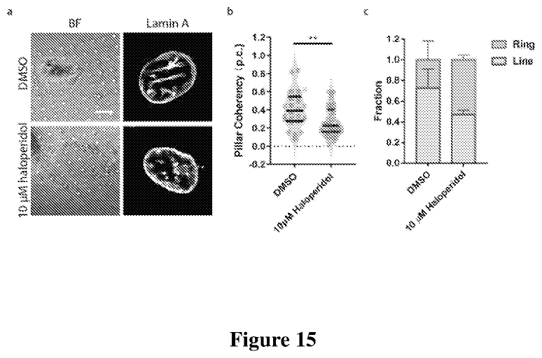

The ability to specifically identify high malignant cells in a mixed cell population can further benefit the evaluation of anti-metastatic drugs. With more than 90 percent of cancer mortality is caused by cancer metastasis, identifying drugs specifically targeting the metastasis-prone cells emerges as a new strategy of cancer therapy. However, the development of anti-metastasis drug is a daunting yet challenging task as metastasis only develops from a subset of cells and is difficult to evaluate using conventional methods probing the whole cell population, such as western blot, transwell migration, and matrigel invasion assays. In comparison, nanopillar-guided subnuclear deformation effectively reveal the cell heterogeneity with single cell resolution, where line deformations specifically indicating high malignant cell with fast migration rate. Therefore, it is hypothesized that the conversion of line patterns of high malignant cells in response to drug treatment can be used to indicate the effectiveness against metastasis (as illustrated in FIG. 9a). As a proof of concept, the inventors examined a reported anti-metastatic drug, curcumin, and compared the deformation changes of MCF-7 and MDA-MB-231 on nanopillars in response to it. As shown in FIG. 9b, the high-malignant MDA-MB-231 cells exhibited significantly less line deformation, while low-malignant MCF7 showed no significant pattern changes. Upon quantification, both decreased pillar coherency values (DMSO, 0.4218±0.1305, n=34 pillars; curcumin, 0.2426±0.1570, n=52 pillars) (FIG. 9c) and decreased fractions of line-deformation cells (DMSO, 0.9333±0.1155, n=27 cells; curcumin, 0.2646±0.2695, n=30 cells) (FIG. 9d) are observed for MDA-MB-231 after curcumin treatment. No significant change in the anisotropy of induced nuclear deformation is found in MCF-7 cells (FIG. 9c-d). Consistently, MCF-7 cells do not show significant changes in migration speed (p value=0.7440) in response to the curcumin treatment, while MDA-MB-231 cells exhibit a lower migration rate (p value=0.0363) (FIG. 9e-f) after the same treatment. Concentration dependency was further characterized, where both the pillar coherency value and fraction of line-deformation cells responded sensitively to as low as 1 μM (FIG. 14). Besides curcumin, another reported anti-metastatic drug, haloperidol, was evaluated and similar responses as shown in FIG. 15 were obtained, which further validated that the anisotropy of nanopillar-guided subnuclear deformation can be an effective indicator for anti-metastatic drug evaluation.

### Conclusion

In summary, the results demonstrate that subnuclear shape irregularities in high-malignant cancer cells can be effectively guided by designed nanopillar arrays, which further enables quantitative evaluation of cancer heterogeneity and assessment of anti-metastatic drug responses with single cell resolution. The ability to localize and quantitatively characterize subnuclear shape irregularities in cancer cells opens up a new angle to study the connection between nuclear biology and cancer development, and provide a new technology for nuclear grading in cancer diagnosis and therapeutic development.

### Example 3

### Fabrication and Characterization of Nanopillar Arrays

SiO2 nanopillar arrays were fabricated via electron-beam lithography (EBL) and anisotropic dry etching. The surface was firstly rinsed with acetone and isopropyl alcohol (IPA). Thin layers of polymethylmethacrylate (PMMA) (MicroChem) and conductive polymer, AR-PC 5090.02 (Allresist), were then spin-coated on the cleaned chip respectively. EBL (FEI Helios NanoLab 650) was subsequently conducted to write different patterns, which decides the diameter and pitch of the nanopillars, on the chip surface. PMMA at the exposed areas was dissolved in the development solution (3:1 isopropanol:methylisobutylketone), followed by the chromium (Cr) deposition by thermal evaporation (UNIVEX 250 Benchtop). Cr masks at the written areas remained at the chip surface after lift-off with acetone, and nanostructures were fabricated through reactive ion etching with a mixture of CF₄ and CHF₃ (Oxford Plasmalab 80). SEM characterization (FEI Helios NanoLab 650) of the nanostructures was carried out after nm Cr deposition.

### Cell Culture and Drug Treatment

Nanopillar arrays were cleaned and sterilized by air plasma and UV exposure before cell culture. The nanochip was then coated with fibronectin (tug/ml, Sigma-Aldrich) for 30 minutes under 37° C. The SK-N-SH cells were seeded on the nanostructures after coating and grown in Eagle's Minimum Essential Medium (MEM) supplemented with 2 mM L-glutamine (Gibco), 1% MEM Non-Essential Amino Acids (NEAA) (Gibco), 10% fetal bovine serum (FBS) (Life Technologies) and 100 U ml⁻¹ penicillin and 100 mg ml⁻¹ streptomycin (Life Technologies) in a standard incubator at 37° C. with 5% CO₂. Drug treatment was done after overnight incubation, the SK-N-SH cells on nanostructures are treated with Etoposide (ETOP) (Sigma), Doxorubicin (DOX) (Sigma) or Dimethyl sulfoxide (DMSO) (Sigma). After 24-hour incubation, the treated cells and untreated cells were fixed with 4% Paraformaldehyde (PFA) Solution in PBS (Boster biological technology AR1068) for 15 minutes for subsequent immunostaining.

### Immunostaining

The nuclear morphology in SK-N-SH cells were visualized by immunostaining lamin A and lamin B1. The lamin A and vimentin level in cells were characterized by immunostaining lamin A and vimentin. Cells were cultured on nanopillars and incubated overnight before fixation with pre-warmed 4% Paraformaldehyde (PFA) in PBS (Boster biological technology AR1068) for 15 minutes at room temperature. After washing with PBS for three times, the cells were permeabilized in 0.5% Triton X-100 (Sigma) in PBS for 15 minutes, followed by blocking with 5% bovine serum albumin (BSA) (Sigma) in PBS for 1 hour. Subsequently, samples were incubated with primary antibodies (anti-lamin A, Abcam, ab26300; anti-lamin B1, Abcam, ab16048; anti-vimentin, Sigma, V5255.) at 1:400 dilution at room temperature for 1 hour or 4° C. overnight. The samples were again washed with PBS for three times and then incubated with secondary antibodies (chicken anti-rabbit IgG Alexa 488, Invitrogen, A21441; anti-mouse IgG Alexa 555, Cell Signaling Technology, 4409s) at 1:600 dilution at room temperature for 1 hour. After washing with PBS three times, the samples were stained with DAPI (Sigma), phalloidin (Cytoskeleton, Inc) or cellmask (Invitrogen) in different experiments.

### Confocal Imaging

Fluorescence imaging and live cell imaging were done by confocal microscopy (Zeiss LSM 800 with Airyscan or a spinning disc confocal microscope (SDC) that is built around a Nikon Ti2 inverted microscope equipped with a Yokogawa CSU-W1 confocal spinning head). Specifically, nuclear morphology of individual cells and the cells in transwell experiments were captured in the Zeiss LSM 800 with Airyscan. The live cell imaging and subsequent fluorescence imaging were done by SDC and the image acquisition and processing was controlled by MetaMorph (Molecular Device) software.

### Quantification of Nanopillar-Guided Subnuclear Irregularities

The guided subnuclear irregularities were characterized by measurement of their anisotropicity. Firstly, one image with the sharpest guided features in one stack was selected for subsequent characterization. Square masks (2.75 μm*2.75 μm, 11 pixels*11 pixels) were manually drawn based on the nanopillar location in the brightfield channel. After thresholding the cropped images of lamin A channel using the masks, the coherency, an indicator for anisotropy property, of the nanopillar-guided nuclear features was measured using orientationJ (a plugin function in ImageJ).

### Transfection

For Lap2b-GFP and lamin A/C-EGFP transfection, SK-N-SH cells were firstly starved in the Opti-MEM (Gibco) medium for 30 mins at 37° C. The transfection mixture was prepared by mixing 1 μg plasmid and 1.5 μl Lipofectamine 3000 (Life Technologies) in Opti-MEM and incubated for 20 mins at room temperature. After 4-hour incubation with the transfection mixture, the Opti-MEM medium was replaced with culture medium and the cells were allowed to recover overnight before imaging.

### Wound Healing Assay

SK-N-SH cells were first cultured in 35 mm dishes until approximately 90% confluent. Scratches were then made in the confluent monolayer of cells using a sterile 200-μl pipette tip, and replaced the culture medium. Brightfield microscopic pictures were taken of the same field at 0 hour and 24 hour. Migration rate was measured by the difference in the closure area within the same time period using Image J.

### Transwell Assay

The SK-N-SH cells were seeded on the top membrane with 5 μm pore size of the transwell device (Corning) and maintained in the culture medium without serum. Complete medium with 10% FBS (Life Technologies) was used as the chemoattractant and placed in the lower chamber. After incubation for 2 hours, the cells were fixed and stained, the distribution lamin A level of cells were examined under confocal microscopy.

### Statistical Analysis

Welch's t tests (unpaired, 2 tailed, not assuming equal SD) were used to evaluate the significance. All tests were performed using Prism (GraphPad Software). Data are presented as mean±SEM or mean±SD as stated in the figure captions. All experiments were repeated at least twice.

### Results and Discussion:

Nanopillar-Guided Subnuclear Patterns Associate with the Heterogeneity of Lamin A Expression Level in SK-N-SH Cells

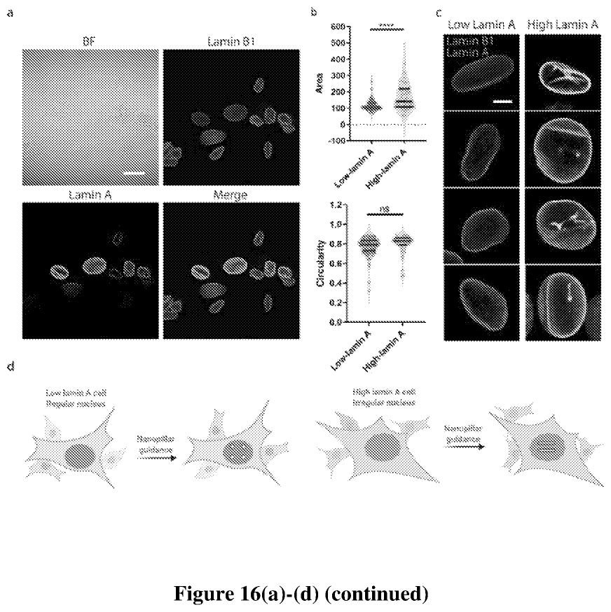

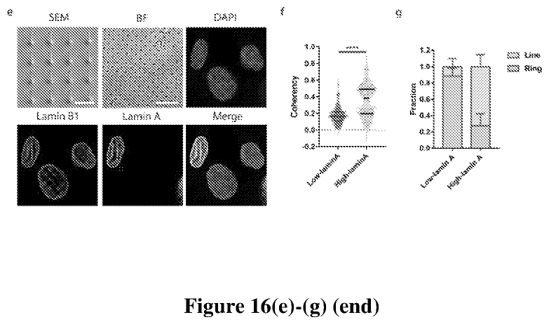

Neuroblastoma cells are known to have different expression levels of lamin proteins that critically impact nuclear morphology and deformability. Here SK-N-SH cells were taken as a model system and probe, firstly, the heterogeneous expression levels of lamin A and lamin B1 via immunostaining Lamin B1 is known to be constitutively expressed in all neuroblastoma cells, while lamin A levels vary significantly and are more associated with differentiated cells. As shown in FIG. 16a, all the cells expressed lamin B1 as expected, but only part of them showed detectable lamin A levels. More interestingly, the nuclear morphology showed a strong correlation with the endogenous level of lamin A, where high-lamin A cells displayed an abnormal nuclear morphology with obvious grooves and invaginations in contrast with low-lamin A cells that normally show a clean nuclear contour (FIG. 16a). However, when characterized using conventional nuclear morphometric parameters like nuclear area and circularity (FIG. 16b), the two populations are not always distinguishable (nuclear area: 115.2±29.72, N=89 cells (low); 165.5±73.13, N=44 cells (high), p value<0.0001. circularity: 0.7720±0.0926, N=89 cells (low); 0.7974±0.0992, N=44 cells (high), p value=0.1595). More importantly, the subnuclear irregularities shown in high-lamin A cells but not in low-lamin A ones (FIG. 16c) are overlooked in the conventional methods that target the overall morphology of the entire nucleus instead of the subnuclear features.

Taking advantage of the nanopillar-guided subnuclear deformation reported earlier, it was investigated whether the guided patterns on nanopillars can effectively reflect the neuroblastoma heterogeneity. As illustrated in FIG. 16d, SK-N-SH cells are classified into low lamin A and high lamin A groups to evaluate their subnuclear features on nanopillars separately. For effective guidance similar to earlier works, arrays of nanopillars with 500 nm in diameter, 3 μm in pitch and 1.5 μm in height on quartz coverslips were fabricated using electron-beam lithography and reactive ion etching, as the scanning electron micrograph (SEM) shown in FIG. 16e. When visualizing the nucleus by immunostaining both lamin B1 and lamin A, it is interesting to see that the cells with low lamin A showed clear ring deformation; while those high in lamin A displayed aligned lines across nearby nanopillars, suggesting a nanopillar-guided alignment of their subnuclear irregular features (FIG. 16e). By characterizing the orientation of nanopillar-guided features using the Orientation J plug-in in ImageJ, a quantified coherency value on each nanopillars was obtained. The coherency ranges from 0 to 1. 0 refers to the fully isotropic features without preferred orientation, suggesting fewer irregularities; while 1 refers to completely anisotropic features with a dominant orientation angle, indicating more irregularities. Comparing cells with low and high lamin A, it is obvious that high-lamin A cells showed a significantly higher pillar coherency value (0.3525±0.1838, n=48 pillars) than low-lamin A cells (0.1727±0.0961, n=122 pillars) (FIG. 16f), consistent with the fact that high-lamin A cells generally have more subnuclear shape irregularities than low-lamin A ones. More interestingly, two subpopulations with different coherency values can be observed in high-lamin A cells according to the two regions with high probability density (pillar coherency˜0.2 and 0.5 respectively) (FIG. 16f). This implies that the heterogeneity exists even within the high-lamin A cells with one subpopulation showing high degree of subnuclear shape irregularities while the other is similar to the low-lamin A cells. By averaging the pillar coherency values in each cell, cell coherency values as a single-cell readout for quantitative analysis were obtained. Setting coherency value at 0.3 as a cutoff for differentiating cells with dominant isotropic ring or anisotropic line deformations, it was found that most of low-lamin A cells (0.885±0.101, n=65 cells) did not show nanopillar-guided abnormal nuclear features. But the majority of high-lamin A cells (0.722±0.147, n=24 cells) exhibited aligned subnuclear irregularities on nanopillars (FIG. 16g).

### Heterogeneity of High-Lamin A Cells Probed by Nanopillar-Guided Subnuclear Patterns

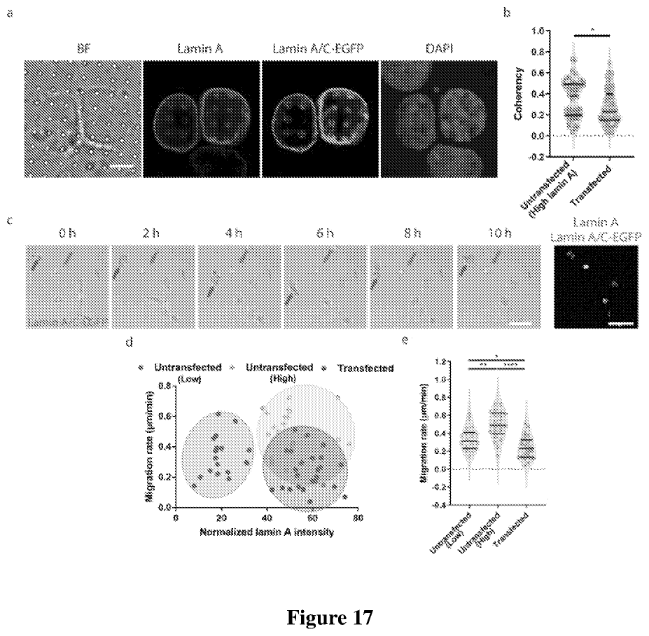

Earlier studies have shown that transient overexpression of lamin A often leads to decreased cell migration. Interestingly, the opposite trend was observed. Higher migration capacity is seen in cells with endogenously high-lamin A level. It raises the question whether such inconsistency is caused by the heterogeneity within the population of high-lamin A cells. As shown in FIG. 16f, two subpopulations among high-lamin A cells can be clearly differentiated based on their nanopillar-guided deformation coherency. So it was hypothesized that the migratability can be more faithfully reflected by the deformation pattern on nanopillars. To prove this, the identified high-lamin A cells were compared with and without transient overexpression of lamin A/C-EGFP (FIG. 17a). Interestingly, the cells with lamin A/C overexpression generate isotropic ring deformation on nanopillars which was observed to give slow migration in breast cancer cells. Statistical analysis further showed a significant decrease (p value=0.0230) of deformation coherency in the transfected cells (0.2735±0.1703, n=62 pillars), so that no more distinguishable subpopulation with higher coherency is left comparing to non-transfected cells (0.3525±0.1838, n=48 pillars) (FIG. 17b). It strongly implies that the different motility in high-lamin A cells is better correlated with deformation coherency on nanopillars, where cells with low coherency, i.e. ring deformation on nanopillars, are slow to migrate.

To verify the effect of lamin A/C overexpression on cell motility, live cell imaging was performed overnight to track the migration of both untransfected and transfected cells, and lamin A level of individual cells were probed by immunostaining at the last time point (FIG. 17c). By correlating the migration rate with lamin A level in the individual cells, it was found that non-transfected cells with endogenous high lamin A level tend to migrate faster than those with low lamin A level (FIG. 17d), which is in agreement with the results shown in FIG. 16. In comparison, most of the transfected cells with overexpressed high-lamin A migrate slower than those with endogenous high-lamin A without transfection (FIGS. 17d and e). More interestingly, the transfected cells (0.2407±0.1275 μm/min, n=24 cells) even migrate slower compared with untransfected cells with endogenously low lamin A level (0.3382±0.1310 μm/min, n=18 cells) (FIG. 17e), suggesting the strong impact of lamin A overexpression on cell motility, which is consistent with the coherency measurement on nanopillars. These results demonstrated the nanopillar-probed deformation coherency reveals better heterogeneity among neuroblastoma cells, and also correlates with one of the key indicators of metastasis, the cell migration speed.

Subnuclear Deformation Patterns on Nanopillar Arrays Correlates with EMT

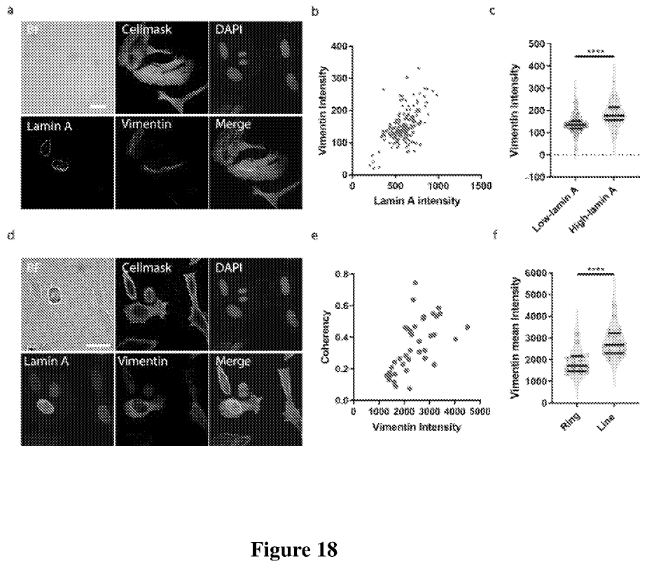

It has been well established that cancer cells typically acquire a migratory phenotype via epithelial-to-mesenchymal transition (EMT) throughout the metastatic progression. It was hypothesized that EMT is responsible for the increased cell motility and invasiveness in the high-lamin A cells. Vimentin was selected among all the EMT markers in this study due to its reported contribution in preserving nuclear integrity. On the flat surface, vimentin level shows a strong dependence with lamin A level, as obvious vimentin filaments were observed in cells with high lamin A, whereas negligible vimentin signal can be found in low lamin A cells (FIG. 18a). In agreement with the observation, vimentin intensity is positively correlated with lamin A level in neuroblastoma cells (FIG. 18b-c). On nanopillar arrays, strong vimentin signal was found in cells with aligned deformation patterns across nearby pillars, while weak vimentin signal was observed in cells showing subnuclear ring features (FIG. 18d). Coherency measurement further revealed a monotonic correlation with the vimentin intensity in the same cell (FIG. 18e). Notably, cells showing dominant anisotropic line deformations (i.e. averaged cell coherency above 0.3) have a higher vimentin than cells exhibiting isotropic ring-like feature (i.e. averaged cell coherency less than 0.3) (FIG. 18f). Interestingly, this suggests that the nanopillar-guided deformation pattern is a valuable indicator of EMT along metastasis development.

### Assessing Anti-Cancer Drug Effect Via Subnuclear Anisotropicity on Nanopillars

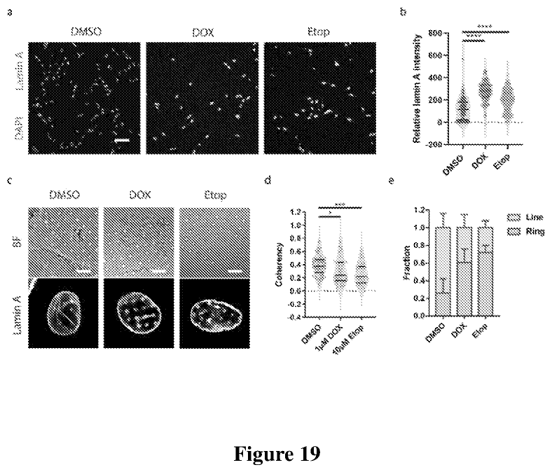

A growing demand for probing heterogeneity in neuroblastoma cells comes from the anti-cancer drug development, as the precise characterization of the drug response among different subpopulations may facilitate the identification of drugs more effectively targeting the metastatic cells to improve prognosis and prevent relapse. Here two drugs widely used for the treatment of neuroblastoma, doxorubicin (DOX, 1 μM) and etoposide (Etop, 10 μM), were chosen. When neuroblastoma cells with various levels of endogenous lamin A were treated with either DOX or Etop for 24 h, it was interesting to observe that most of the cells that remained attached on the surface exhibit significantly high lamin A level (FIG. 19a-b). It may be that the drug treatment upregulated the lamin A expression. However, it is not clear whether individual cells with potentially upregulated-lamin A after drug treatment form similar ring deformation patterns as the over-expressed ones shown in FIG. 19a, or the line patterns in endogenous high-lamin A cells. Their nanopillar-guided subnuclear deformation patterns upon treatment were examined. Strikingly, compared to the more common line features observed in the control group, cells treated with either DOX or Etop tend to develop more isotropic ring features on nanopillar arrays despite the high lamin A level (FIG. 19c). As a result, the pillar coherency of the treated cells (DOX: 0.3057±0.1862, n=38 pillars; Etop: 0.2606±0.1564, n=44 pillars) is significantly lower (DOX: p value=0.0344; Etop: p value=0.0002) than that of the DMSO control group (0.3853±0.1448, n=47 pillars) (FIG. 19d). Consistently, the fraction of cells with dominant isotropic ring features and low deformation coherency increases upon anti-cancer drug treatment (DMSO: 0.262±0.160, n=30 cells; DOX: 0.607±0.152, n=22 cells; Etop: 0.772±0.079, n=24 cells) (FIG. 19e). Since the isotropic ring fractions are more associated with less EMT marker and slower migration as shown earlier, their increased fraction upon drug treatment suggests that although the high-lamin A cells survives the drug treatment, their metastasis ability is significantly reduced. Together, this demonstrates that the deformation coherency can be employed as a highly sensitive indicator for anti-cancer drug efficacy in neuroblastoma cells.

### Conclusion

In this study, it was demonstrated that nanopillar-guided subnuclear deformation patterns can be used as an effective marker to characterize the heterogeneity in neuroblastoma cells. On top of various lamin A expression levels widely reported in neuroblastoma, cells with similar lamin A levels were further differentiated into subpopulations depending on the orientation coherency of their deformation features on nanopillars. High deformation coherency was found to exist at a higher percentage in high-lamin A cells with faster migration speed, and positively correlated with EMT marker increase. These results demonstrated that the risk stratification of individual neuroblastoma cells can be achieved via quantitative assessment of the subnuclear irregularities guided on nanopillar arrays. More importantly, the subtle response of drug-resistant high-lamin A cells can be sensitively reflected through the changes in deformation patterns and can be quantitatively readout using coherency value measurement. It is envisioned that this nuclear irregularities-based risk stratification in neuroblastoma cells opens up new avenues to improve cancer therapy.

1. A method of grading the nuclear morphology of a tumor cell, the method comprising:
a) culturing the tumor cell on an array comprising a plurality of nanopillars; and
b) assessing a deformation pattern of the nucleus to provide an indication of the nuclear morphology of the tumor cell.

2. The method of claim 1, wherein the morphology is a nuclear morphology.

3. The method of claim 1, wherein the array comprising the plurality of nanopillars is capable of inducing nanometer scale deformation pattern in the nucleus of the tumor cell.

4. The method of claim 1, wherein the tumor is a cancer.

5. The method of claim 1, wherein the morphology provides an indication of the malignancy potential of the tumor cell.

6. The method of claim 1, wherein an isotropic deformation pattern indicates low malignancy of the tumor cell.

7. The method of claim 1, wherein linear or anisotropic deformation pattern indicates high malignancy of the tumor cell.

8. The method of claim 1, wherein the nanopillars have a diameter of about 0.1 microns to 2 microns.

9. The method of claim 1, wherein the nanopillars have a pitch of about 0.5 micron to about 8 microns.

10. The method of claim 1, wherein the nanopillars have a height of about 0.5 micron to about 20 microns.

11. The method of claim 1 wherein the array is coated with a biomolecule.

12. The method of claim 1, wherein step b) comprises staining and visualising the nucleus by microscopy.

13. A method of grading a sample of tumor cells, comprising:
a) obtaining an image of one or more cells of the sample captured on an array of nanopillars; and
b) determining a nanopillar-induced deformation profile of the nucleus in the one or more cells.

14. The method according to claim 13, wherein determining the deformation profile comprises determining an angular distribution of orientations in a plurality of regions of interest (ROIs) of the image, each ROI containing a nanopillar.

15. The method according to claim 14, comprising determining a coherency in each ROI.

16. The method according to claim 14, wherein determining the deformation profile comprises detecting one or more quantifiable morphological parameters.

17. A method of predicting the progression of a tumor in a subject, the method comprising:
a) culturing a cell sample on a surface comprising a plurality of nanopillars; and
b) assessing the degree of nuclear deformation of cells of the cell sample to provide an indication of the progression of the tumor in the subject.

18. The method of claim 17, wherein the method comprises obtaining a cell sample from the subject prior to step a).

19-23. (canceled)
